(19) United States
(12) Patent Application Publication
(10) Pub. No.: US 2011/0160740 A1
(43) Pub. Date: Jun. 30, 2011

(54) Tissue Removal in The Paranasal Sinus and Nasal Cavity

(75) Inventors: Joshua Makower, Los Altos, CA (US); John Y. Chang, Mountain View, CA (US); Eric Goldfarb, Belmont, CA (US); Bernard H. Andreas, Redwood City, CA (US); Thomas R. Jenkins, Oakland, CA (US); Nicholas Sherman, Waxhaw, NC (US); Ronda M. Heiser, San Jose, CA (US); Hung V. Ha, San Jose, CA (US); John H. Morriss, San Francisco, CA (US)

(73) Assignee: ACCLARENT, INC., Menlo Park, CA (US)

(21) Appl. No.: 12/974,834

(22) Filed: Dec. 21, 2010

Related U.S. Application Data
(60) Provisional application No. 61/290,341, filed on Dec. 28, 2009.
(60) Provisional application No. 61/298,800, filed on Jan. 27, 2010.

(51) Int. Cl. A61B 17/26; A61M 29/00

(52) U.S. Cl. 606/115; 606/192

(57) ABSTRACT

Sinusitis, tumors, infections, fractures and other disorders of the paranasal sinuses, are diagnosed and/or treated using minimally invasive approaches. Access devices may be used to facilitate insertion of working devices such catheters, balloon catheters, dilation catheters, tissue cutting or remodeling devices, suction or irrigation devices and biopsy devices.

### CROSS REFERENCE TO RELATED APPLICATIONS

This application claims the benefit of Provisional Application Ser. No. 61/290,341, filed Dec. 28, 2009 and Provisional Application Ser. No. 61/298,800, filed Jan. 27, 2010, the contents of which are incorporated by reference.

### FIELD OF THE INVENTION

The present invention relates generally to medical devices, systems and methods and more particularly to minimally invasive devices, systems and methods for performing therapies within the paranasal sinuses.

### BACKGROUND

Chronic sinusitis is a widespread and debilitating medical condition that effects over thirty million people annually in the U.S. Ear, nose and throat (ENT) doctors typically treat sinusitis with nasal steroids and/or antibiotics, but oftentimes these medications to not work and patients must then decide to simply endure their symptoms or undergo painful surgery. The symptoms of chronic sinusitis, include headaches, difficulty breathing, feelings of intense pressure in the head and face, toothache, congestion, and runny nose and often significantly reduce a sufferer's quality of life.

Functional endoscopic sinus surgery (FESS) is currently the most common type of surgery used to treat chronic sinusitis. In a typical FESS procedure, a rigid endoscope is inserted into the nostril along with one or more rigid surgical instruments, and the instruments are used to remove bone and mucosal tissue in the nasal cavity and the openings into the paranasal sinuses, to enlarge such openings and thus hopefully improving sinus drainage and mitigating sinusitis symptoms. In most FESS procedures, the natural ostium (e.g., opening) of at least one paranasal sinus is surgically enlarged to improve drainage from the sinus cavity. FESS procedures can be effective in the treatment of sinusitis and for the removal of tumors, polyps and other aberrant growths from the nose, however, removing significant amounts of tissue from the nasal cavities causes bleeding and post-operative pain and scarring and typically necessitates nasal packing and painful debridement (scar removal), all of which is difficult for the patient and requires additional physician visits.

FESS procedures usually include the surgical removal or modification of normal anatomical structures, in order to access the openings into one or more paranasal sinus. For example, in many FESS procedures, a total uncinectomy (e.g., removal of the uncinate process in the nasal canal) is performed at the beginning of the procedure to allow visualization and access of the maxillary sinus ostium and/or ethmoid bulla. In general, as more tissue is removed, post-operative recovery for the patient becomes more longer and more painful. At the same time, it may sometimes be advantageous to remove some tissue, such as polyps or bony growths, when performing a sinus surgery.

More recently, new devices, systems and methods have been devised to enable a less invasive procedure for dilating openings into paranasal sinuses to mitigate or alleviate chronic sinusitis. In one example, a balloon dilation catheter is advanced into and expanded within a natural paranasal sinus ostium to dilate the ostium. A system for performing such a Balloon Sinuplasty™ procedure is provided by Acclarent, Inc., of Menlo, Calif. (www.acclarent.com). Examples of this and other methods, devices and systems are described, for example, in U.S. patent application Ser. Nos. 10/829,917, entitled Devices, Systems and Methods for Diagnosing and Treating Sinusitis and Other Disorders of the Ears, Nose and/or Throat; 10/944,270, entitled Apparatus and Methods for Dilating and Modifying Ostia of Paranasal Sinuses and Other Intranasal or Paranasal Structures; 11/116,118, entitled Methods and Devices for Performing Procedures Within the Ear, Nose, Throat and Paranasal Sinuses; and 11/150,847, entitled Devices, Systems And Methods Useable For Treating Sinusitus, all of which are hereby incorporated fully by reference.

In some cases, ENT physicians both remove tissue as in a traditional FESS procedure and also perform one or more balloon dilations in the same patient. Ideally, such a combined tissue-removal/balloon-dilation procedure would remove a minimal amount of tissue while still providing a desired outcome for the patient. Also ideally, such a procedure would be relatively straightforward for the physician. At least some of these objectives will be met by the present invention.

### SUMMARY

The present disclosure describes methods, devices and systems for treating the paranasal sinuses. In various embodiments, the paranasal sinus (or sinuses) treated may include one or more of the maxillary, frontal, ethmoid and/or sphenoid sinuses. Also in various embodiments, methods, devices and systems of the present disclosure may involve removal of tissue and/or substances from one or more sinuses and/or the nasal cavity, dilation of one or more openings into one or more paranasal sinuses, dilation of other areas/structures within the nasal cavity, or any suitable combination of these procedures. In embodiments where an opening to a paranasal sinus is dilated, this opening may be either a natural paranasal sinus ostium or a manmade opening. In some embodiments, a natural paranasal sinus ostium of one sinus and a manmade opening of another sinus may be dilated using the same system. In some embodiments, an area such as but not limited to the frontal sinus outflow tract, the osteomeatal complex or the like may be dilated. Any suitable combination of these or other procedural steps described herein is contemplated.

In one aspect of the present invention, a method for treating a paranasal sinus in a patient may involve: advancing a dilating device along a guide device into the patient's head; dilating an opening into the paranasal sinus, using the dilating device; removing the dilating device from the head while leaving the guide in the patient's head; advancing a tissue removal device along the guide into the paranasal sinus; and removing tissue from at least one of the paranasal sinus, a different paranasal sinus, or a nasal cavity of the patient using the tissue removal device. In various embodiments, the guide device may be a guide catheter with a lumen, a guidewire, or a combination of a guide catheter and a guidewire. In one embodiment, the dilating device is a balloon catheter.

In one embodiment of the method, removing the tissue involves suctioning the tissue into the tissue removal device and cutting off the suctioned tissue using a cutter of the tissue removal device. In various embodiments, removing the tissue may involve removing tissue from the opening into the paranasal sinus, removing tissue from within the paranasal sinus, or a combination of both.

In one embodiment, dilating the opening involves dilating at least one of a frontal sinus ostium and a frontal sinus outflow tract, and removing the tissue involves removing tissue from the frontal sinus outflow tract. In one embodiment, dilating the opening involves dilating a natural paranasal sinus ostium of the paranasal sinus, and removing the tissue involves removing bone fragments. In another embodiment, dilating the opening involves dilating a natural paranasal sinus ostium of the paranasal sinus, and removing the tissue involves removing at least part of an ethmoid sinus. Any suitable type of tissue may be removed using this method, including but not limited to polyps, mucosal tissue, cysts, bone fragments, bone and/or mucocysts. Similarly, in removing the tissue any suitable apparatus may be employed, including but not limited to a morcellator, a snare, a combined snare/cutter, a filter, a radiofrequency cutting and/or coagulating device, a contractable mesh device, a balloon configured with blades, a bone cutter assembly, a tube with openings in combination with a cutter, a high pressure spray device, and/or a forceps-grasper assembly.

In another aspect of the present invention, a method for treating a paranasal sinus in a patient may include: advancing a guide catheter into the patient's head; advancing a guidewire through the guide catheter and through an opening into the paranasal sinus; advancing a balloon dilation catheter over the guidewire through the guide to position a balloon of the catheter in the opening; dilating the opening by expanding the balloon; removing the balloon, leaving at least the guidewire in place; advancing a tissue removal device over the guidewire into the patient's head; removing tissue from at least one of the paranasal sinus, a different paranasal sinus and a nasal cavity of the patient with a tissue removal device; and removing the tissue removal device and the guidewire from the patient's head.

In another aspect, a method for treating a paranasal sinus in a patient may include: advancing a guide device into the patient's head; advancing a tissue dilation and removal device along the guide device to position a dilator of the device in an opening into the paranasal sinus; dilating the opening by expanding the dilator; removing tissue from the paranasal sinus using the tissue dilation and removal device; and removing the tissue dilation and removal device and the guide device from the patient's head.

In yet another aspect, a system for treating a paranasal sinus in a patient may include: a guide device for guiding one or more devices into the patient's head to treat the paranasal sinus; a dilation device for dilating an opening into the paranasal sinus and configured for passage along the guide device; and a tissue removal device for removing tissue from inside the paranasal sinus and configured for passage along the guide device.

As mentioned above, in various embodiments, the guide device may include a guide catheter with a lumen, a guidewire, or both. In one embodiment, the dilating device may be a balloon catheter. In various embodiments, the tissue removal device may include one or more of the following: suction substructure, a cutter, a snare, and/or a radiofrequency energy delivery member. Also in various embodiment, the tissue removal device may be configured to remove tissue such as but not limited to polyps, mucosal tissue, cysts, bone fragments, bone and/or mucocysts.

In another aspect, a system for treating a paranasal sinus in a patient may include: a guide device for guiding one or more devices into the patient's head to treat the paranasal sinus; and a combined tissue dilation and removal device for dilating an opening into the paranasal sinus and removing tissue from the paranasal sinus, wherein the tissue dilation and removal device is configured for passage along the guide device.

Further aspects, details and embodiments are described below and in the accompanying drawings.

### BRIEF DESCRIPTION OF THE DRAWINGS

FIG. 1 shows a schematic diagram of the general working environment of an example of a system for catheter-based minimally invasive sinus surgery being used to perform a sinus surgery on a human patient.

FIG. 1A shows a magnified view of region 1A of FIG. 1 showing a system for catheter-based minimally invasive sinus surgery of a human patient.

FIG. 1B shows a perspective view of a treatment tray for catheter-based minimally invasive sinus surgery of a human patient.

FIG. 2 shows a perspective view of a guide catheter comprising a plastically deformable (malleable) region.

FIG. 3 shows perspective view of an embodiment of a guide catheter comprising a straight hypotube.

FIG. 3A shows a crossection of the guide catheter of FIG. 7 through plane 3A-3A.

FIG. 4A shows a coronal section of the paranasal anatomy showing a method of accessing a maxillary sinus ostium using the guide catheter of FIG. 2F.

FIG. 4B shows a sagittal section of the paranasal anatomy showing the method of FIG. 8G to access a maxillary sinus ostium using the guide catheter of FIG. 8F.

FIG. 5 shows a perspective view of a set of devices to dilate or modify ostia or other openings.

FIG. 6 shows a perspective view of an embodiment of a balloon catheter comprising a sizing balloon and a dilating balloon.

FIG. 6A shows a crossectional view through the plane 6A-6A of FIG. 6.

FIGS. 6B-6D show the various steps of dilating an anatomical opening using the balloon catheter in FIG. 6.

FIG. 7 shows a perspective view of a cutting device comprising cutting jaws.

FIG. 7A shows a perspective view of the distal region of the cutting device of FIG. 7 wherein the cutting jaws are closed as seen from the distal end of the cutting device.

FIG. 7B shows a perspective view of one embodiment of the cutting jaws of the cutting device of FIG. 7.

FIG. 7C shows a crossectional view of the cutting device in FIG. 7 through cutting plane 7C-7C.

FIG. 8A shows a perspective view of an alternate embodiment of a device comprising cutting or gripping jaws.

FIG. 8B shows a perspective view of the device of FIG. 8A wherein the cutting or gripping jaws of the cutting device are in a closed configuration.

FIG. 9A shows a perspective view of an embodiment of an ostium enlarger and/or microshaver.

FIG. 9B shows one embodiment of the device of FIG. 9A being used to remove tissue or matter.

FIG. 9C shown another embodiment of the device of FIG. 9A being used to shave tissue or matter.

FIG. 9D is an exploded view of the device of FIG. 9C.

FIG. 10A-10C show a suction and snare device and various steps of employing the device to capture and remove biological substances.

FIGS. 11A-11C depict a suction and morcellator device and various steps of employing the device to capture and remove biological substances.

FIGS. 12A-12C depict a suction grasper device and various steps of employing the device to capture and remove biological substances.

FIGS. 13A-13C depict a tissue capture suction device and various steps of employing the device to capture and remove biological substances.

FIGS. 14A-14C depict a tissue capture device including a capture vial and various steps of employing the device to capture and remove biological substances.

FIGS. 15A-15C depict a capture screen device and various steps of employing the device to capture and remove biological substances.

FIGS. 16A-16E depict a capture and cutting balloon device and various steps of employing the device to capture and remove biological substances.

FIGS. 17A-17D show a spin cutter device and various steps of employing the device to capture and remove biological substances.

FIGS. 18A-18C show a back cutter device and various steps of employing the device to capture and remove biological substances.

FIGS. 19A-19C show a balloon and cutter device and various steps of employing the device to capture and remove biological substances.

FIGS. 20A-20C show a spinning shaped cutter device and various steps of employing the device to capture and remove biological substances.

FIGS. 21A-21C show a high pressure flushing device and various steps of employing the device to capture and remove biological substances.

FIGS. 22A-22C depict an ultrasonic agitation device and various steps of employing the device to capture and remove biological substances.

FIGS. 23A-23D depict a forceps grasper device and various steps of employing the device to capture and remove biological substances.

FIGS. 24A-24E show various devices for scrubbing and swabbing devices.

FIGS. 25A-25E depict an approach for treating a paranasal sinus.

### DETAILED DESCRIPTION

The following detailed description, the accompanying drawings and the above-set-forth Brief Description of the Drawings are intended to describe some, but not necessarily all, examples or embodiments of the invention. The contents of this detailed description, the accompanying drawings and the Brief Description of the Drawings do not limit the scope of the invention in any way.

A number of the drawings in this patent application show anatomical structures of the ear, nose and throat. In general, these anatomical structures are labeled with the following reference letters:
Nasal Cavity NC
Nasopharynx NP
Frontal Sinus FS
Frontal Sinus Ostium FSO
Ethmoid Sinus ES
Ethmoid Air Cells EAC
Sphenoid Sinus SS
Sphenoid Sinus Ostium SSO
Maxillary Sinus MS
Maxillary sinus ostium MSO
Mucocyst MC
Eustachian tube ET
Cochlea
Tympanic cavity TC
Middle turbinate MT
Inferior turbinate IT
Uncinate UN

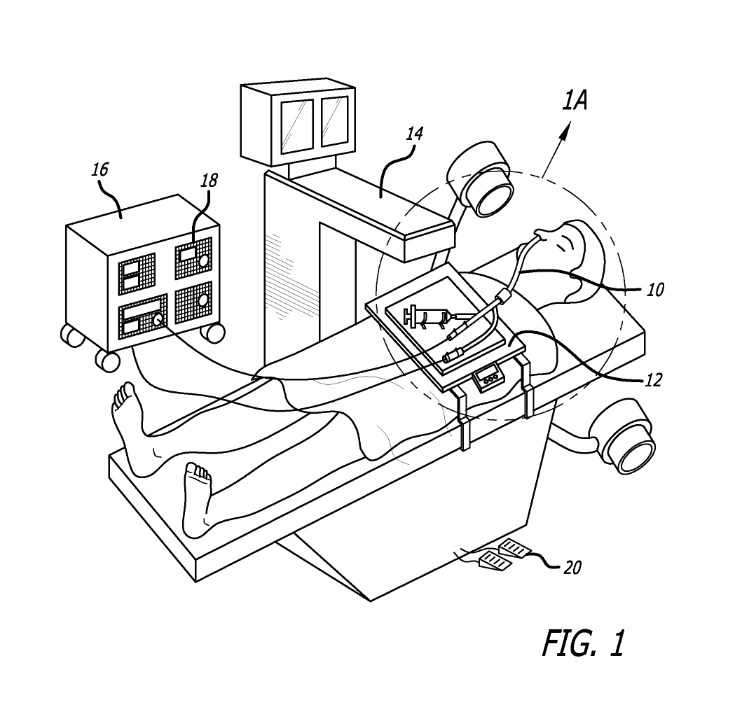

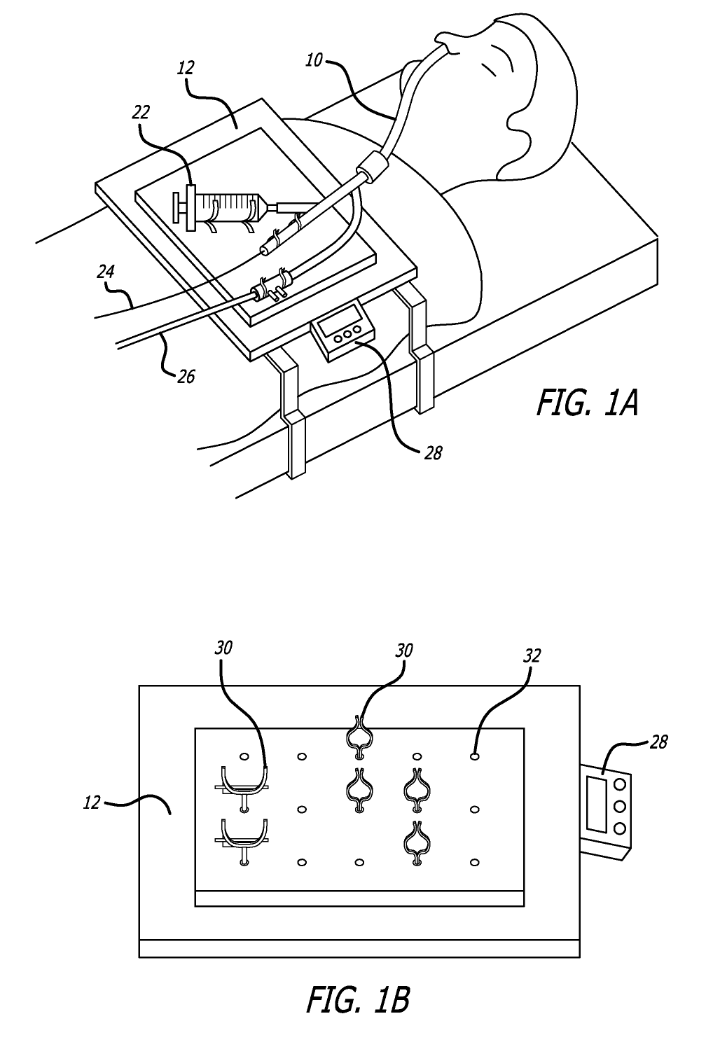

FIG. 1 shows a schematic diagram of the general working environment of an example of a system for catheter-based minimally invasive sinus surgery being used to perform a sinus surgery on a human patient. The human patient is treated by a working device 10. Working device 10 may be connected to one or more auxiliary devices located on a treatment tray 12. A C-arm fluoroscope 14 provides fluoroscopic visualization of anatomical regions during the procedure. An instrument console 16 comprising one or more functional modules 18 may also be present. Examples of functional modules that can be used with the invention are:

1. Suction pump for delivering a controlled amount of negative pressure or vacuum to a suction device,

2. Irrigation pump to deliver saline, antibiotic solution or other suitable irrigation medium,

3. Power module to supply power to drills or other electrical devices,

4. Storage modules for storing instruments, medications etc.,

5. Energy delivery module to provide radiofrequency, laser, ultrasound or other therapeutic energy to a surgical device,

6. Fluoroscope, MRI, CT, Video, Endoscope or Camera or other imaging modules to connect or interact with devices used during various diagnostic or therapeutic procedures,

7. Display module e.g. a LCD, CRT or Holographic screen to display data from various modules such as an endoscope, fluoroscope or other data or imaging module,

8. Remote control module to enable an operator to control one or more parameters of one or more functional modules 18,

9. Programmable Microprocessor that can store one or more operation settings for one or more functional modules 18 etc., and

10. Stabilization device for holding various apparatuses during the procedure which may include a stabilization arm, table, clip, intranasal or extranasal inflatable support or robotically controlled apparatus,

11. Rotary drive module for rotating rotatable device such as a drill or auger (e.g., a motor having a rotation drive shaft or drive cable attached thereto.

One or more functional modules 18 may be connected to the working device 10. Instrument console module 16 can be controlled by console control means 20, e.g. a foot pedal controller, a remote controller etc. Instrument console 16 may be fitted with wheels to enable an operator to change the position of the instrument console 16 in an operating area. In one embodiment, instrument console module 16 and C-arm fluoroscope 14 are integrated in a single unit.

FIG. 1A shows a magnified view of region 1A of FIG. 1 showing a system for catheter-based minimally invasive sinus surgery of a human patient. In FIG. 1A, a balloon catheter is used as an example of working device 10. Working device 10 has attachments for a variety of auxiliary devices such as a balloon inflation syringe 22, a guidewire 24 and a suction or irrigation tube 26. Working device 10 and the auxiliary devices may be detachably attached to treatment tray 12. Treatment tray 12 may comprise one or more treatment tray controllers 28 to control one or more treatment parameters. Treatment tray 12 may comprise one or more storage modules to store devices used during a surgery e.g. irrigation bottles, swabs etc.

FIG. 1B shows a perspective view of a treatment tray for catheter-based minimally invasive sinus surgery of a human patient. Treatment tray 12 comprises one or more device holders 30 to detachably hold devices during the surgery. In one embodiment, device holders 30 are detachably attached to device holder slots 32 on treatment tray 12. Thus the position of device holders 30 on treatment tray 12 can be changed by removing a device holder 30 from a device holder slot 32 and transferring to a new device holder slot 32.

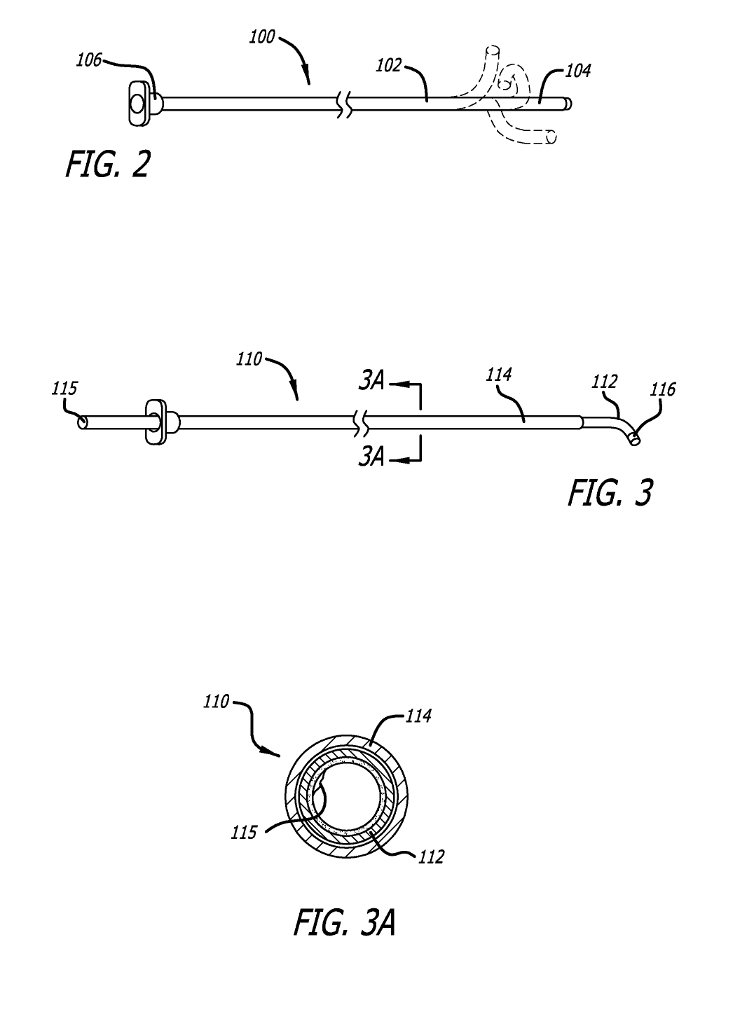

Any diagnostic or therapeutic device disclosed herein may comprise one or more malleable regions. For example, FIG. 2 shows a perspective view of a guide catheter comprising a plastically deformable (malleable) region. Such a guide catheter 100 can be employed to place a tissue or biological substance removal device at a target site in a paranasal sinus. Guide catheter 100 comprises a shaft 102 comprising a malleable region 104 located on distal region of shaft 102. Shaft 102 may comprise stiffening elements e.g. a braid, hypotube etc. Malleable region 104 may comprise malleable metallic tubes, rods (e.g. rods embedded in shaft 102 etc.), wires etc. Examples of metals that can be used for constructing malleable region 104 are malleable stainless steel, fully annealed stainless steel, copper, aluminum etc. Guide catheter 100 further comprises a threaded luer 106 located on proximal end of shaft 102. In this example, malleable region 104 is located on distal end of guide catheter 100. Malleable region 104 can also be located on proximal region or any other intermediate region on shaft 102. Shaft 102 may also comprise more than one malleable regions. Such a design comprising one or more malleable regions can be used for any of the devices mentioned herein such as catheters with working elements, guide catheters, guide catheters with a pre-set shape, steerable guide catheters, steerable catheters, guidewires, guidewires with a pre-set shape, steerable guidewires, ports, introducers, sheaths or other diagnostic or therapeutic devices.

FIG. 3 shows perspective view of an embodiment of a guide catheter comprising a straight hypotube. This structure can also be used to place a tissue or substance removal device within a sinus cavity for accomplishing desired therapies. Guide catheter 110 comprises a tubular element 112 and a hypotube 114 attached to the external surface of tubular element 112. Suitable materials for constructing hypotube 114 are Stainless Steel 304, Nitinol etc. In one embodiment, hypotube 114 is annealed to the external surface of tubular element 112. Tubular element 112 can be made from a variety of materials including Pebax, HDPE etc. Tubular element 112 may comprise a braid or a jacket. In an embodiment, tubular element 112 comprises a lubricious coating 115 on its inner surface. The lubricious coating 115 can be made of suitable lubricious materials such as Teflon. In an embodiment, tubular element 112 comprises a bent or angled region near the distal end of tubular element 112. The bent or angled region may enclose an angle from 0 degrees to 180 degrees. Further this bent or angled region may be further bent out of plane to present a compound three-dimension end shape. Hypotube 114 can be malleable or substantially stiff. A malleable hypotube can be used in situations where the guide catheter 110 has to be bent or distorted to optimize its shape to conform to a patient's anatomy. Examples of materials that can be used to make a malleable hypotube are malleable stainless steel, fully annealed stainless steel, copper, aluminum etc. A substantially stiff hypotube can be used in situations where extra support is needed for introduction or removal or devices through guide catheter 110. Examples of materials that can be used to make a substantially stiff hypotube are Stainless Steel 304, Nitinol etc. Hypotube 114 may be bent to a two-dimensional or three-dimensional shape. Distal tip of tubular element 112 may comprise a radio-opaque marker 111 e.g. a standard radio-opaque marker band. The proximal region of tubular element 112 comprises a threaded luer.

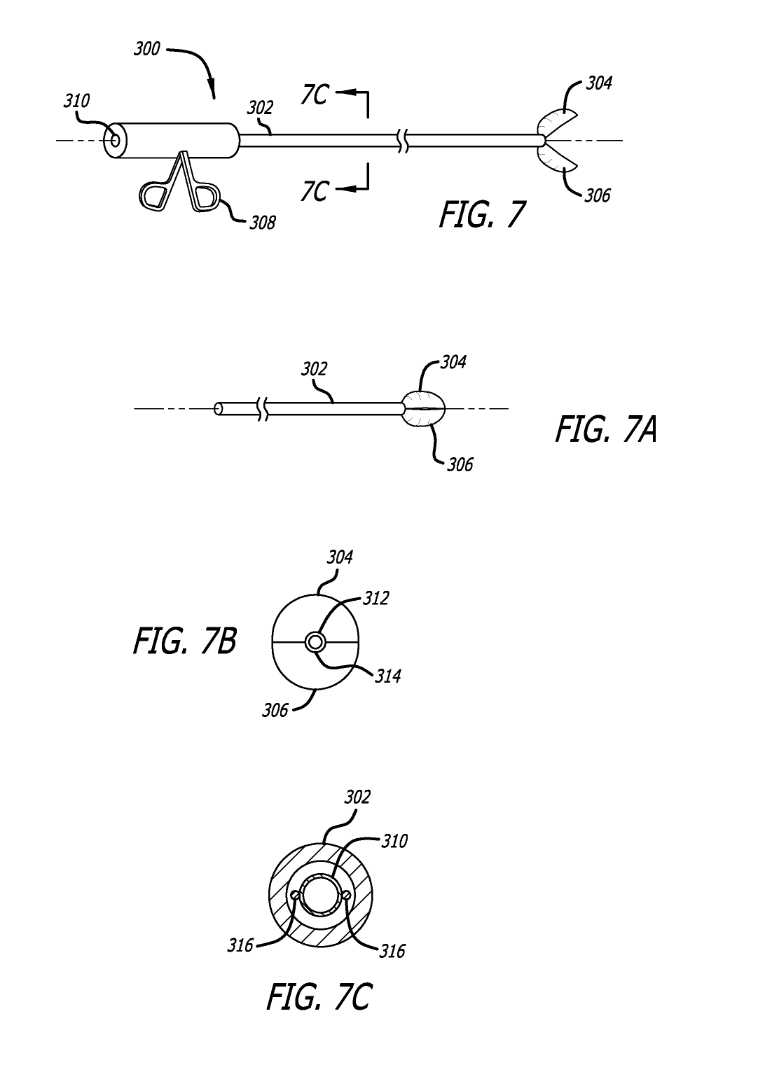

FIG. 3A shows a crossectional view of guide catheter 110 of FIG. 7 through plane 3A-3A. The crossection of guide catheter 110 shows an outer hypotube 114 enclosing a tubular member 112 which in turn comprises a lubricious coating 115 located on the inner surface of tubular member 112.

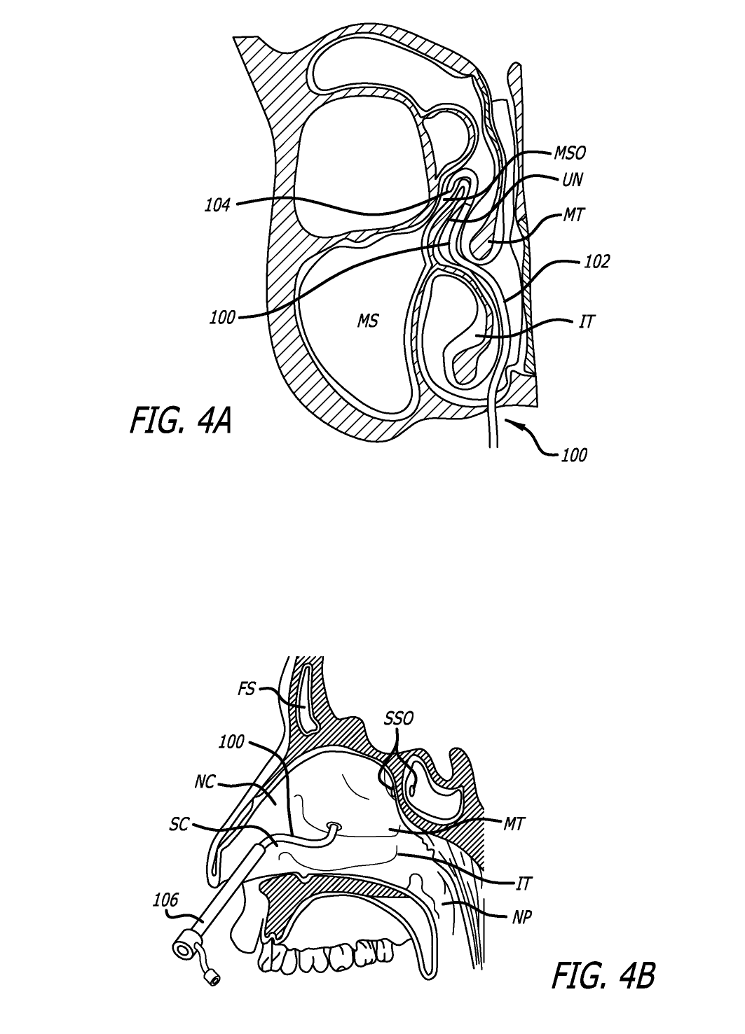

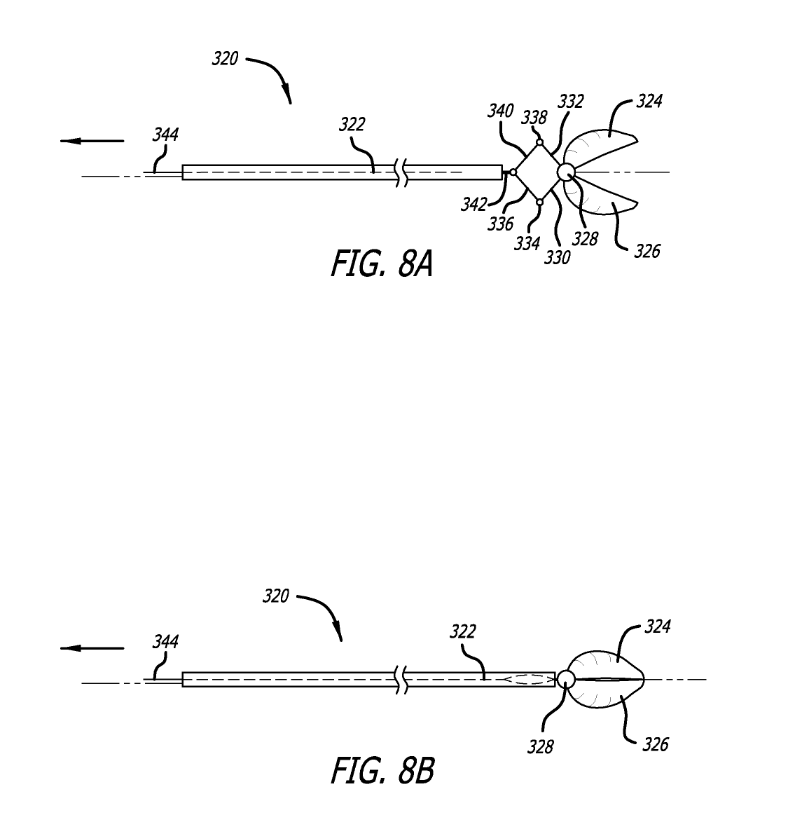

FIG. 4A depicts a coronal section of the paranasal anatomy showing a method of accessing a maxillary sinus ostium using guide catheter 100 of FIG. 2. Guide catheter 100 is introduced through a nostril and advanced in the paranasal anatomy such that atraumatic tip 104 is located inside or adjacent to a maxillary sinus ostium MSO. Proximal bent, curved or angled region 102 allows guide catheter 100 to be positioned around the inferior turbinate IT. Similarly, distal bent, curved or angled region 104 allows guide catheter 100 to be positioned around the middle turbinate MT. A guidewire or a suitable diagnostic or therapeutic device may then be introduced through the lumen of guide catheter 100 into the maxillary sinus MS. FIG. 8B shows a sagittal section of the paranasal anatomy showing the method of FIG. 8G to access a maxillary sinus ostium using guide catheter 100 of FIG. 2.

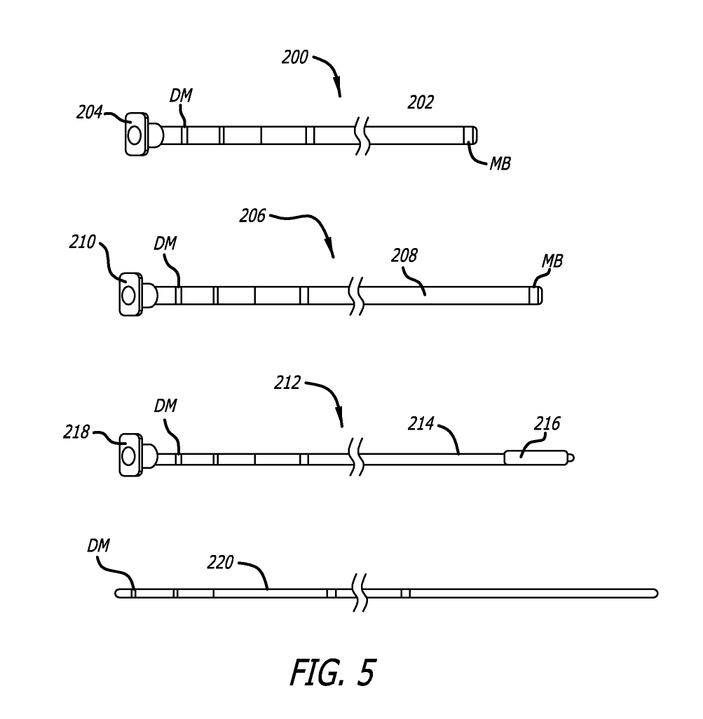

FIG. 5 shows a perspective view of a set of devices to dilate or modify ostia or other openings in the sinuses or other body cavities. Guide catheter 200 comprises a shaft 202 comprising a threaded luer 204 at proximal end of shaft 202. Distal end of shaft 202 comprises a radio-opaque marker band MB to enable the physician to identify the tip of shaft 202 in a fluoroscopic image. The distal end of shaft 202 may be substantially straight or may comprise one or more bent or angled regions. One or more distance markings DM may also be located on the shaft 202. An optional subselective catheter 806 may also be present in the set of devices. Subselective catheter 206 comprises a shaft 208 comprising a threaded luer 210 at the proximal end of shaft 208. Inner diameter of shaft 208 is smaller than inner diameter of shaft 202. Distal end of the shaft 208 comprises a radio-opaque marker band MB to enable the physician to identify the tip of shaft 208 in a fluoroscopic image. Distal end of shaft 208 may be substantially straight or may comprise one or more bent or angled regions. One or more distance markings DM may also be located on the shaft 208. Working device 212 comprises a shaft 214 comprising a working element 216 located on distal region of shaft 214 and a threaded luer 218 located on proximal end of shaft 214. The working element 216 can be a dilating balloon or can be one or more of a combination of suction or irrigation devices, needles, polypectomy tools, brushes, brushes, energy emitting devices such as ablation devices, laser devices, image-guided devices containing sensors or transmitters, endoscopes, tissue modifying devices such as cutters, biopsy devices, devices for injecting diagnostic or therapeutic agents, drug delivery devices such as substance eluting devices, substance delivery implants etc.

In one embodiment of a method, the guide catheter 200 is introduced into a patient's body so that distal end of guide catheter 200 is in the vicinity of an anatomical opening (e.g. an ostium) of an anatomical region (e.g. a paranasal sinus). Thereafter, the guidewire 220 is introduced through guide catheter 200 into the anatomical region e.g. the paranasal sinus. If necessary, guide catheter 200 may be removed and the smaller subselective catheter 206 may be introduced over guide wire 220 into the paranasal sinus. Thereafter, working device 212 is introduced over guidewire 220 into the paranasal sinus and a diagnostic or therapeutic procedure is performed by the working device 212. In another embodiment of a method using the abovementioned set of devices, subselective catheter 206 is omitted from the procedure. Additionally, in yet another approach, larger guide catheter 200 can be introduced over guide wire 220. The working device 212 is then introduced over guidewire 220 into the paranasal sinus and a diagnostic or therapeutic procedure is performed by working device 212. This method embodiment enables a user to introduce larger working device 212 in the anatomical region.

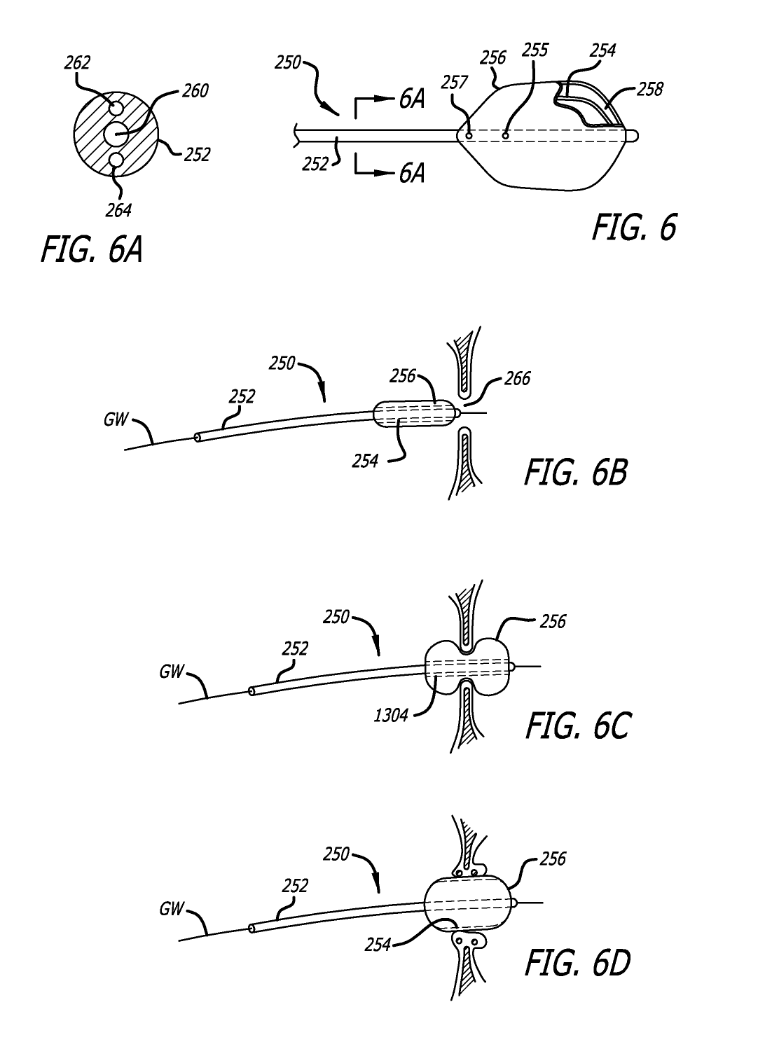

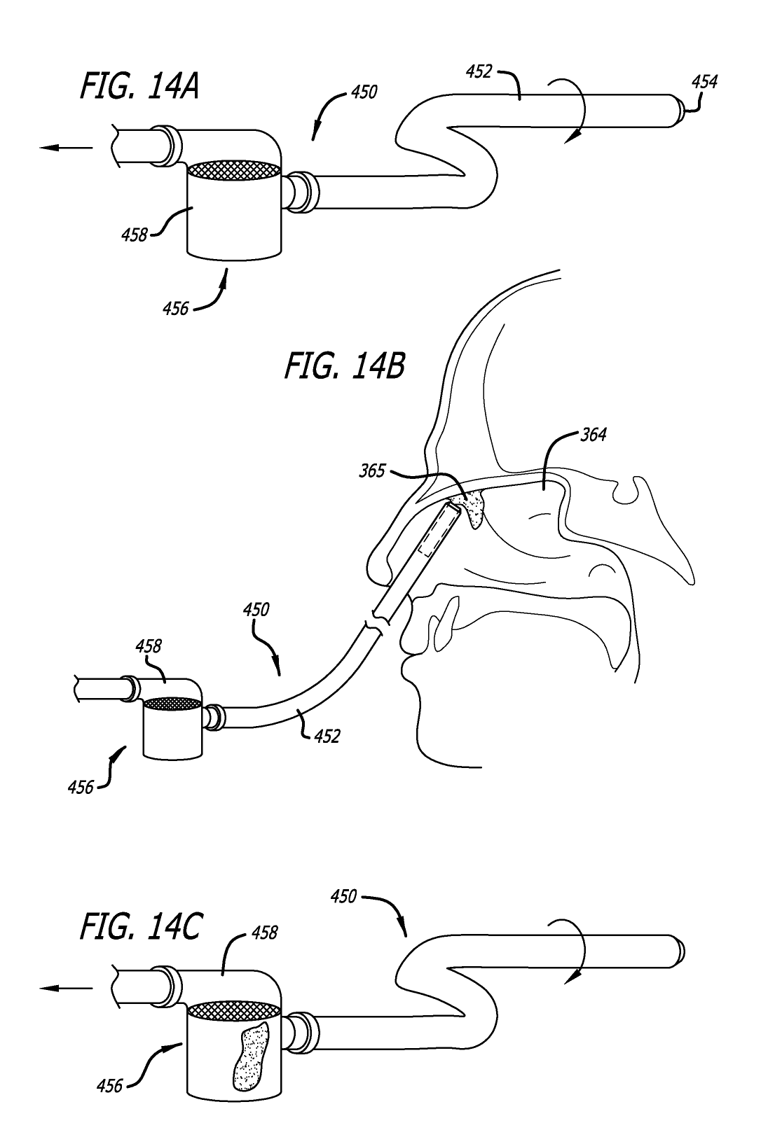

FIG. 6 shows a perspective view of an embodiment of a balloon catheter comprising a sizing balloon and a dilating balloon. The balloon catheter can be used as a treatment modality in combination with tissue or biological substance removal. A portion of the sizing balloon has been removed to show the dilating balloon underneath the sizing balloon. Balloon catheter 250 comprises a shaft 252 and a dilating balloon 254 located on distal region of shaft 252. Dilating balloon 254 can be made of suitable non-compliant materials e.g. polyethylene terephthalate, Nylon etc. Dilating balloon 254 is inflated through a first balloon inflation opening 255. Balloon catheter 250 further comprises a sizing balloon 256 located around dilating balloon 254. Sizing balloon 256 is made from a compliant or semi-compliant material such as crosslinked polyethylene or other polyolefins, polyurethane, flexible polyvinylchloride, Nylon etc. Sizing balloon 256 is inflated through a second balloon inflation opening 257. Dilating balloon 254 and sizing balloon 256 enclose an inter-balloon volume 258. FIG. 6A shows a crossection of the balloon catheter in FIG. 6 through plane 6A-6A. Shaft 252 comprises a guidewire lumen 260, a first inflation lumen 262 that terminates distally in first balloon inflation opening 255 of FIG. 14, and a second inflation lumen 264 that terminates distally in second balloon inflation opening 257 of FIG. 6.

FIGS. 6B-6D show the various steps of dilating an anatomical opening using the balloon catheter in FIG. 6. In FIG. 6B, balloon catheter 250 is introduced over a guidewire GW into an anatomical opening 266 to be dilated. Examples of the types of anatomical openings 266 that may be dilated by this invention include ostia of paranasal sinuses, Eustachian tubes, ostia of lachrymal ducts, etc. Thereafter, in FIG. 6C, sizing balloon 256 is inflated using an imageable inflating medium. Examples of suitable imageable inflating media are saline with a radioopaque contrast agent, carbon dioxide gas etc. Distal region of balloon catheter 250 is subsequently imaged using a suitable imaging modality such as fluoroscopy or X-rays. This enables an operator to accurately estimate the size of anatomical opening 266. Such a balloon catheter is also suited for estimating the diameter of the narrowest region in a tubular anatomical region e.g. a Eustachian tube prior to performing a diagnostic or therapeutic procedure such as balloon dilation. On the basis of information obtained during step 6C, balloon catheter 250 may be repositioned and step 6C repeated if necessary. Thereafter, in step 6D, sizing balloon 256 is deflated. Also in step 6D, dilating balloon 254 is inflated to dilate a target region in anatomical opening 266. Thereafter, dilating balloon 254 is deflated and balloon catheter 250 is withdrawn from anatomical opening 266. In one embodiment, sizing balloon 256 may be reinflated after a balloon dilation procedure to obtain feedback about the performance of the balloon dilation procedure.

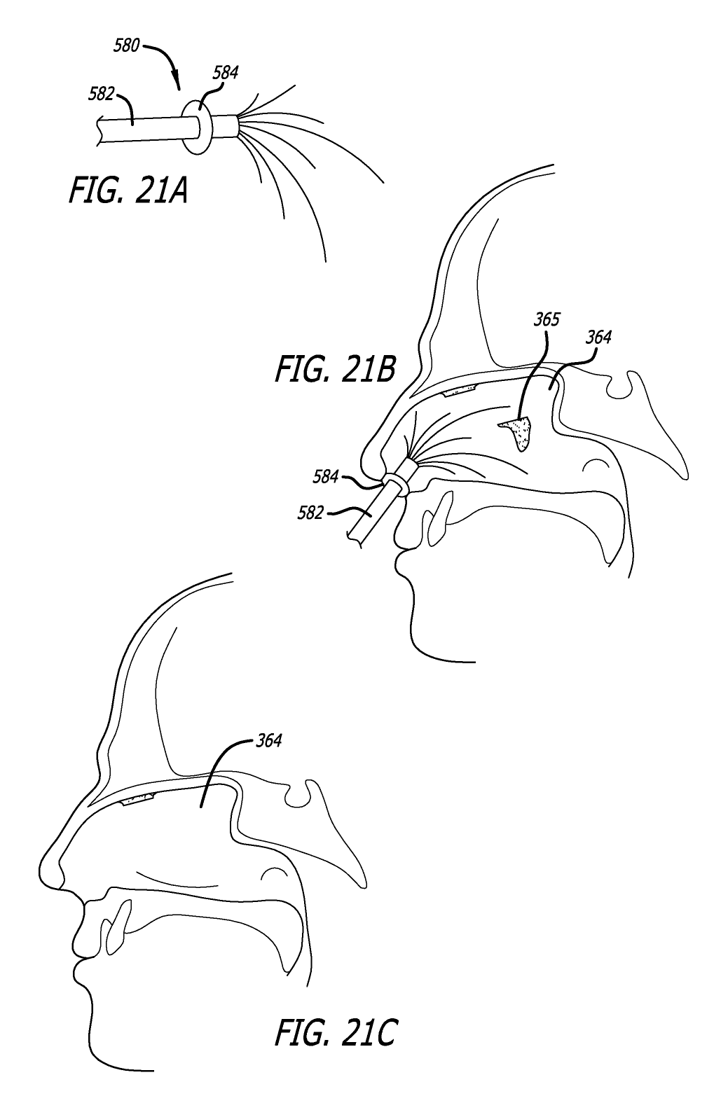

Prior, subsequent or contemporaneously with the implant of a balloon catheter, tissue and/or biological substances can be captured or removed from a treatment site. Such capture and removal devices can be deployed over a guidewire or can embody structure permitting direct placement at the interventional site. FIG. 21 depicts one approach to a cutting device comprising cutting jaws. Cutting device 300 comprises a shaft 302 comprising an upper jaw 304 and a lower jaw 306 located on the distal end of shaft 302. Proximal region of shaft 302 comprises a scissor-like device with handles or other suitable control apparatus 308 that is useable to control the movement of upper jaw 304 and/or lower jaw 306. Upper jaw 304 and lower jaw 306 are hinged together so that they can be opened or closed by scissor handles 308 to bite, grip or cut tissue. In one embodiment, the edges of upper jaw 304 and lower jaw 306 are provided with a series of cutting teeth. Alternately, the edges of upper jaw 304 and lower jaw 306 may be provided with sharp edges, blunt gripping teeth etc. Shaft 302 comprises a lumen 310. This enables cutting device 300 to be advanced over an access device such as a guidewire to access a target anatomical region. Examples of materials that can be used to construct cutting device 300 are stainless steel 304, stainless steel 316, titanium, titanium alloys etc.

FIG. 7A shows a perspective view of the distal region of the cutting device of FIG. 7 wherein the cutting jaws are closed.

FIG. 7B shows a perspective view of one embodiment of the jaws of the cutting device of FIG. 7. Upper jaw 304 comprises an upper jaw notch 312. In one embodiment, upper jaw notch 312 is semicircular in shape. Similarly, lower jaw 306 comprises a lower jaw notch 314. In one embodiment, lower jaw notch 314 is semicircular in shape. This design enables a guidewire to pass through a gap in the distal end of the cutting device 300 even when upper jaw 304 and lower jaw 306 are closed. In another embodiment, a guidewire passes through an opening located on either upper jaw 304 or lower jaw 306. Upper jaw 304 and lower jaw 306 can also be square, ovoid, trapezoidal or circular in shape.

FIG. 7C shows a crossectional view of the cutting device in FIG. 7 through plane 7C-7C. Shaft 302 of cutting device 300 comprises a lumen 310 for an access device such as a guidewire. Shaft 302 further comprises one or more pull wires 316 that connect upper jaw 304 and lower jaw 306 to control apparatus 308. When the control apparatus 308 is moved, pull wires 316 transmit the movement to upper jaw 304 and lower jaw 306 causing them to open or close.

FIG. 8A shows a perspective view of an alternate embodiment of a device comprising cutting or gripping jaws. Cutting device 320 comprises a shaft 322. Distal end of cutting device 320 comprises an upper jaw 324 and a lower jaw 326 that are hinged together at a first hinge 328. Proximal end of upper jaw 324 comprises a first elongate member 330 and proximal end of second jaw 326 comprises a second elongate member 332. The proximal end of first elongate member 330 is connected to a second hinge 334 which in turn is connected to a third elongate member 336. Proximal end of second elongate member 332 is connected to a third hinge 338 which in turn is connected to a fourth elongate member 340. The proximal ends of third elongate member 336 and fourth elongate member 340 are connected by a fourth hinge 332 to pull wire 344 that passes through shaft 322. FIG. 8A shows cutting device 330 wherein the upper jaw 334 and lower jaw 326 are in an open configuration. When pull wire 344 is pulled in the proximal direction, fourth hinge 342 is pulled inside shaft 322. This causes the distal ends of third elongate member 336 and fourth elongate member 340 to come closer to each other. This in turn causes the proximal ends of first elongate member 330 and second elongate member 332 to come closer to each other. This in turn causes upper jaw 324 and lower jaw 326 close. Similarly, pushing pull wire 344 in the distal direction causes upper jaw 324 and lower jaw 326 to open. In one embodiment, cutting device 320 comprises a spring mechanism located between pull wire 344 and shaft 322 that biases upper jaw 324 and lower jaw 326 in an open or closed configuration.

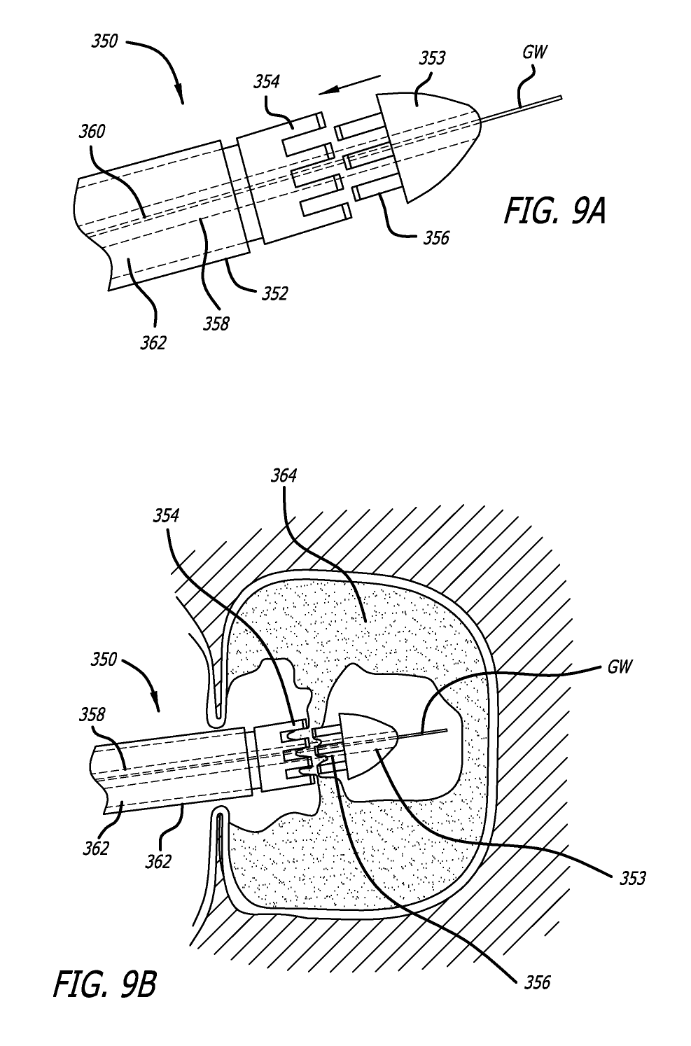

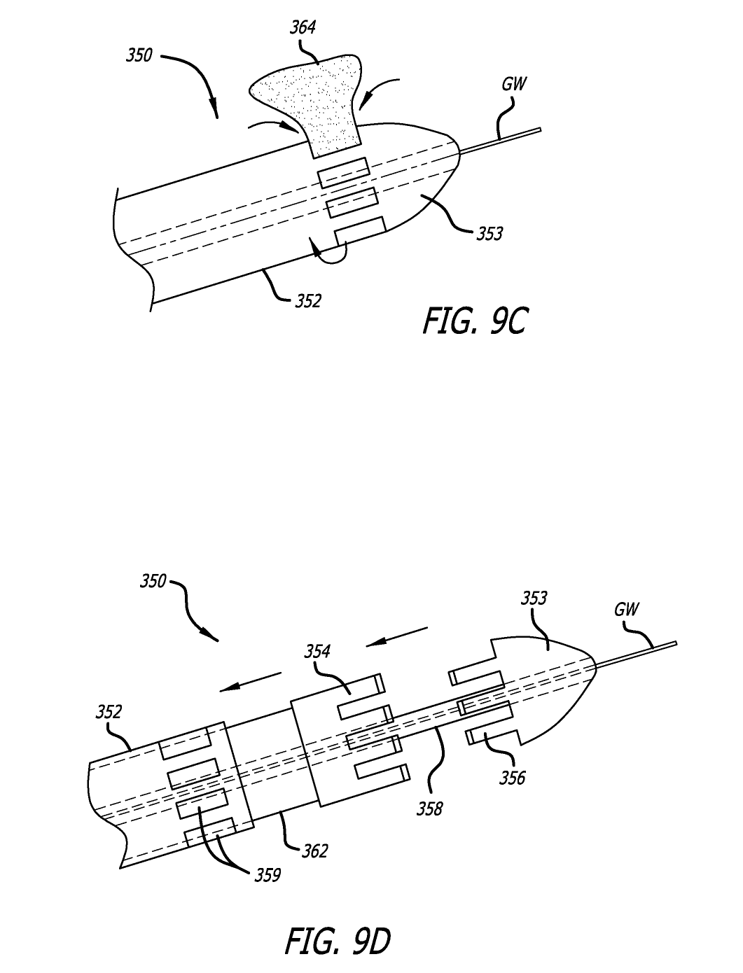

FIG. 8B shows a perspective view of the device of FIG. 9A wherein the jaws of the cutting device are in a closed configuration.

FIG. 9A shows a perspective view of an embodiment of a microshaver or ostium enlarger device 350. Device 350 comprises a proximal portion 352 and a distal portion 353. Proximal portion 352 is hollow and comprises a proximal cutting surface 354 e.g. sharp cutting teeth etc. located on the distal end of proximal portion 352. Distal portion 353 comprises a distal cutting surface 356 e.g. sharp cutting teeth etc. located on the proximal end of distal portion 353. Distal portion 353 is further connected to a pull shaft 358 that encloses a guidewire lumen 360. Guidewire lumen 360 allows microshaver 350 to be introduced over a guidewire GW into a target anatomy. The region between pull shaft 358 and proximal portion 352 encloses a suction lumen 362. Suction lumen 362 can be used to remove solid debris or liquids from the target anatomy by suction. Proximal portion 352, distal portion 353 and pull shaft 358 can be made of suitable biocompatible materials such as stainless steel.

FIG. 9B shows a crossection of a paranasal sinus showing one way in which the device 350 of FIG. 9A may be used to remove tissue or matter. The device 350 is introduced over a guidewire GW into paranasal sinus 364. The device 350 is then positioned such that the tissue or matter is located between proximal cutting surface 354 and distal cutting surface 356. Thereafter, in this embodiment, pull shaft 358 is pulled in the proximal direction. This causes movement of distal region 353 in the proximal direction with respect to proximal portion 352. This in turn forces cylindrical distal cutter 356 to be retracted into the interior of the cylindrical proximal cutter 354, thereby cutting off or breaking tissue or matter that is captured therebetween. Optionally, in this embodiment, the cylindrical distal cutter 356 cylindrical proximal cutter 354 may be rotated relative to the other to further cut or shave tissue. Also, optionally in this embodiment, suction lumen 352 can be used to remove any solid debris or liquids generated during the procedure.

FIGS. 9C and 9D show an example of another way in which the device 350 may be used—i.e., to shave tissue or matter. Examples of anatomical structures that may be shaved by this device 350 include bone, cartilage and soft tissues of Eustachian tubes, turbinates, lachrymal ducts, anatomical openings such as ostia of paranasal sinuses, ostia of lachrymal ducts, etc. and other regions in the ear, nose, throat or mouth. As shown in FIG. 9C, in this embodiment, there need not be a proximally moveable pull shaft 358, but rather the distal cutting surface 356 may remain positioned within the cylindrical proximal cutting surface 354. The cutting surfaces are positioned adjacent to the tissue or matter to be shaved and the cylindrical distal cutter 356 and/or cylindrical proximal cutter 354 is/are rotated to shave the tissue or matter. Suction may be applied through lumen 362 to draw the tissue or matter into slots 359 such that it will be shaved by the rotating proximal cutter 354.

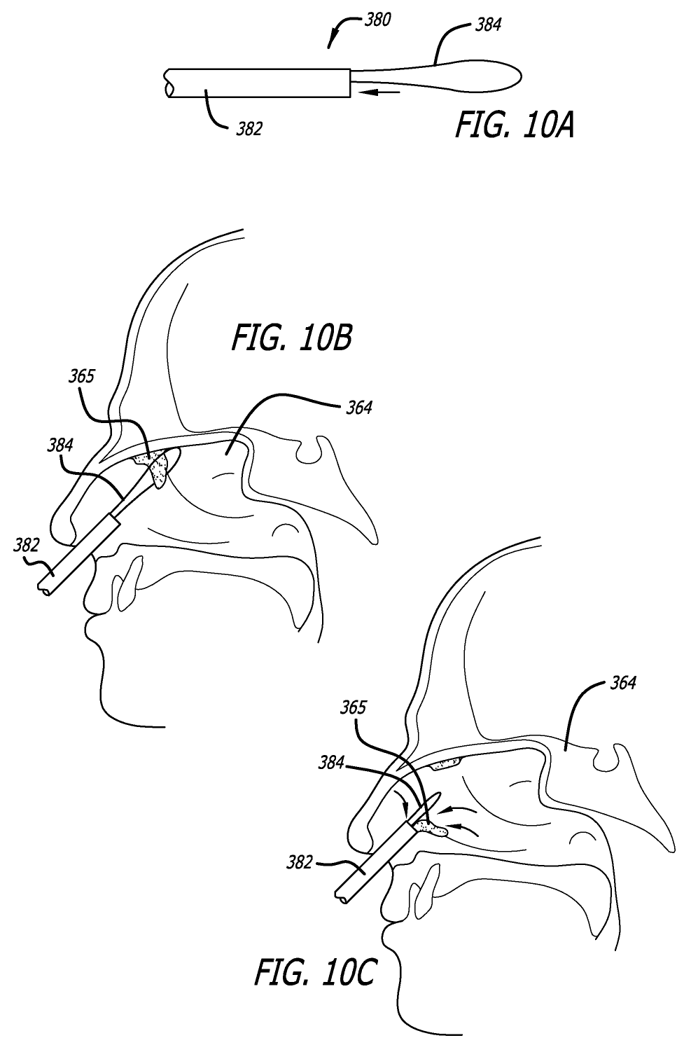

Referring now to FIGS. 10A-C, in one embodiment a tissue removal device 380 may include an elongate tubular member 382 and a snare 384. The tissue removal device 380 may be used to remove tissue from the nasal cavity and/or from within a paranasal sinus prior to, subsequent to, or contemporaneous with the use of dilation catheters such as those depicted in FIG. 6. In various embodiments, tissue removal device 380 may be used to remove tissue in one or more paranasal sinuses, in the nasal cavity, or both. The elongate tubular member 382 may include a proximal end configured to be attached to a device that creates a negative pressure gradient within the tubular member 382. In one embodiment, the elongate tubular member 382 may further include a balloon or other expandable member to allow the device 380 to both remove tissue and dilate an opening or other space in the nasal cavity or paranasal sinuses. In one embodiment, the tissue removal device 380 may be advanced over a guidewire and/or through an introducer or guide catheter, such as those described above in reference to shown FIGS. 2 and 3. In alternative embodiments, the tissue removal device 382 may be advanced without the use of a guide device.

The elongate tubular member 382 may include an internal lumen sized and shaped to accept the snare 384. The snare 384 may be moved longitudinally with respect to the elongate tubular member 380 so that it can be both advanced beyond a distal end of the elongate tubular member 380 and withdrawn completely within the internal lumen of the tubular member 380. In various embodiments, the snare 384 may be either a simple mechanical loop of material such as a wire or may be equipped to transmit RF energy. In the latter embodiments, a proximal end of the snare 384 may be connected to a RF energy transmitting device.

As shown in FIGS. 10B and 10C, the tissue removal device 380 may be advanced into a patient's nasal cavity 364 (and/or one or more paranasal sinuses) to engage tissue or other biological substances. In these and subsequent sets of figures, devices are shown removing tissue from the nasal cavity for ease of illustration. However, in many if not all embodiments, the devices and methods shown in use in such figures may also be used to remove tissue from within one or more paranasal sinuses. At some point during or after advancement of the device 380, the snare 384 may be advanced out of the elongate tubular member 382 and manipulated to snare and cut a target tissue or substance. In a purely mechanical approach, the snare 384 can be placed about the target tissue and withdrawn to cut the tissue from the sinus cavity. Alternatively, RF energy can be transmitted to accomplish the severing of tissue. Contemporaneously with this severing action or subsequent thereto, a suction force is applied via the elongate tubular member 382. The suctioning and withdrawing of the snare 384 then accomplishes the capture of the target substances. The device 380 and severed tissue/substances can then be removed from the interventional site or further severing and collection of material can be performed until the site is cleared as desired.

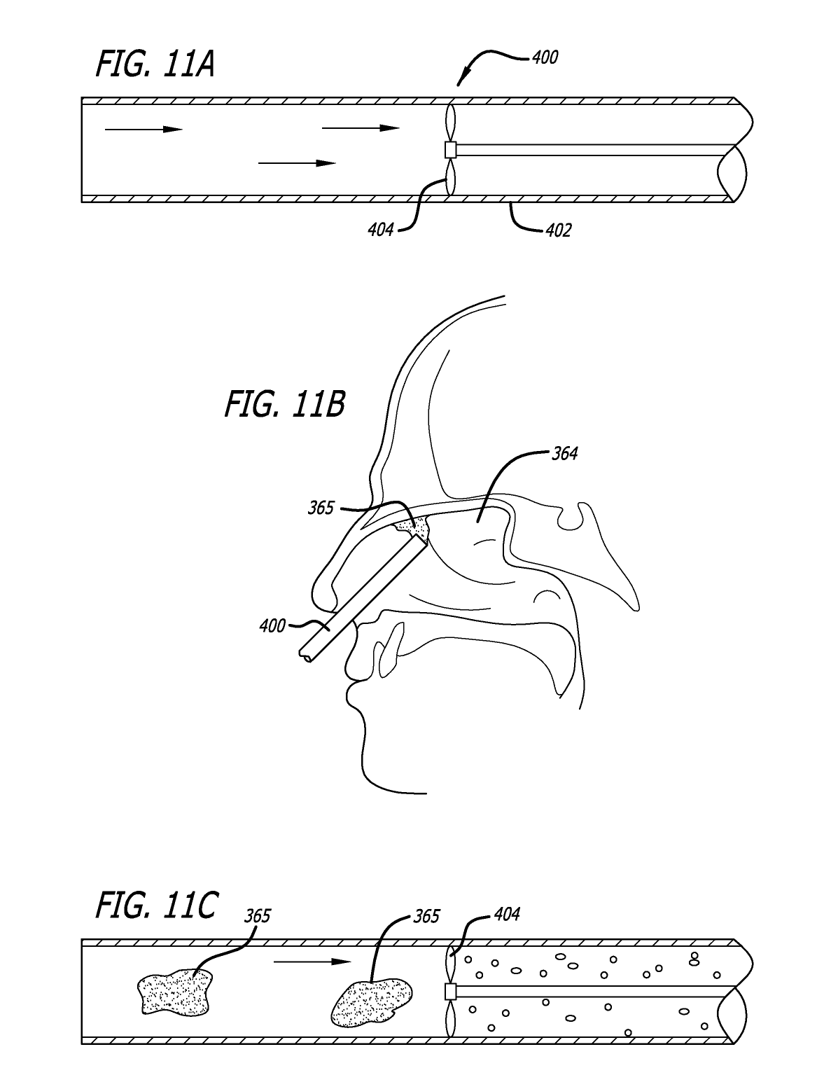

With reference now to FIGS. 11A-11C, in a related approach, a therapy system 400 for removing biological substances from paranasal cavities can include an elongate, tubular, suctioning member 402 having a distal end portion configured with a morcellator 404. Again, balloon dilation or other dilation of paranasal cavities can be conducted along with targeted tissue removal. Also, the elongate tubular member 402 is intended to be connected to a device that creates a suctioning force within a lumen running a length of the elongate tubular member 402. The morcellator 404 can define various sub-assemblies designed to break down, sever or cut biological substances found in a patient's sinuses. In the approach depicted in the drawing figures, the morcellator 404 can include a plurality of blades that rotate about a central hub. A control device can be attached to the morcellator 404 and extend proximally to an operator so that rotation of the morcellator 404 can be effected.

In use, in combination with or separate from balloon dilation of the paranasal cavity, a distal end of the suction and morcellator assembly 400 can be placed as previously described within a paranasal sinus adjacent substances targeted for removal. Suctioning pressure can then be generated within the elongate tubular member 402 to begin the substance collection process. The morcellator 404 is then activated to sever, cut or chop the targeted substances. The targeted biological substances are then withdrawn within the elongate tubular member 402 and can be removed from the patient's sinuses. This procedure can be repeated as necessary to clean out a sinus to a desired degree. Further balloon dilation can also be conducted to fully treat the sinuses.

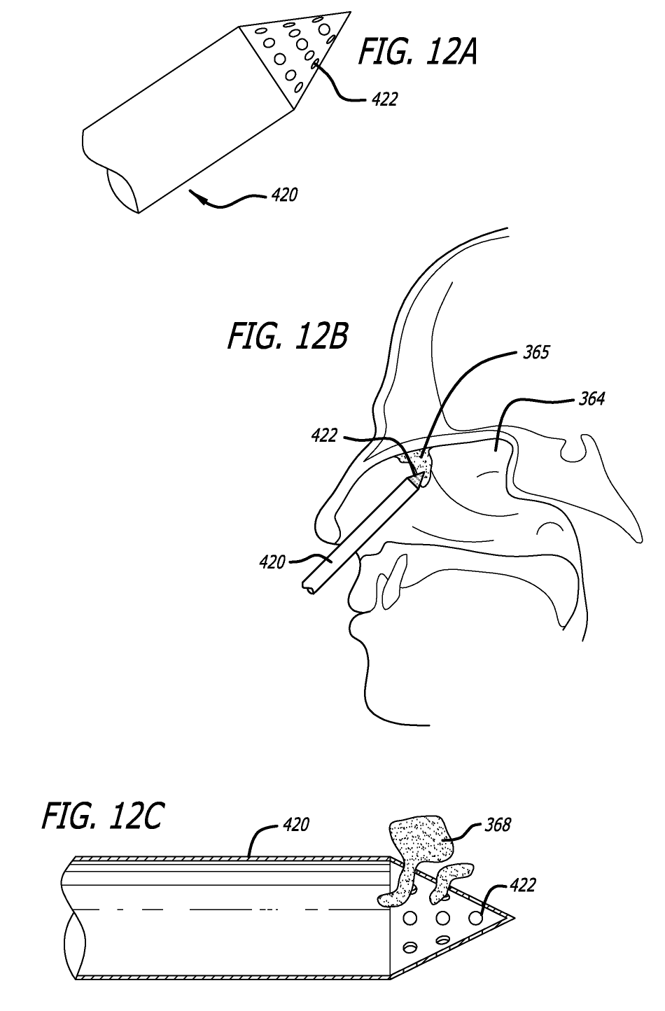

Now turning to FIGS. 12A-12C, another embodiment of a suctioning tubular member 420 is disclosed. Independently or along with balloon dilation of a paranasal sinus opening or other nasal/paranasal area, the suctioning tubular member can be used for therapies for treating the sinuses. As before, the suctioning tubular member 420 can further include a balloon or other expandable member for dilating paranasal sinus anatomy, or a separate catheter can be employed for this purpose. A proximal end of the device 420 is again attached to an assembly that generates a suctioning force. In the present approach, a distal end of the suctioning tubular member 420 is configured with a single limb with a plurality of holes 422 formed therein to adhere to tissue reversibly using suction. As shown in the figures, this relatively less traumatic approach to tissue manipulation can be used to capture and remove tissue 365 or other substances from sinus and/or nasal cavity anatomy.

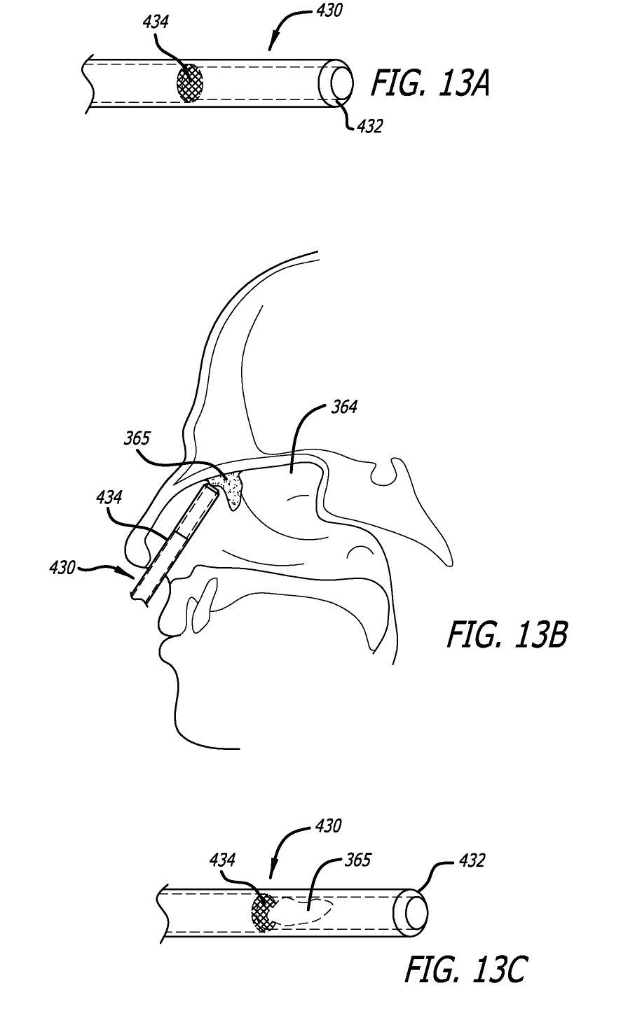

In yet another related approach (FIGS. 13A-13C), a tissue capture suction device 430 having a generally elongate tubular body structure and a distal end including a cutting edge 432 is contemplated to sever, capture or remove tissue from sinus anatomy. This assembly as well can be advanced over a guidewire and/or within a guide catheter or can itself define a guide catheter. Moreover, this device can be used with or independently from a balloon catheter or other structure for dilating paranasal sinus and/or nasal cavity anatomy, or expandable structure can be incorporated directly into the tubular body.

As shown, the cutting edge 432, which extends a full circumference of the tubular body, is defined by a sharp angle between an internal luminal wall of the tubular capture suction device 430 and an outer surface thereof. Various other approaches to cutting surfaces are also contemplated such as sharp edges extending less than a full circumference of the distal tubular portion. The tissue capture suctioning device 430 further includes a filter 434 for registering captured material within the device.

In use, separate or contemporaneously with balloon or other sinus dilation, the tissue capture suctioning device 430 is placed at the intervention site adjacent substances to be collected. By way of a connection to a suction subassembly, suction force is applied while the cutting edge 432 is manipulated to engage and sever the substances targeted for removal. When the device dislodges the substances target for removal, the suctioning force draws the substance within the elongate tubular member, the filter 434 operating to retain the substance in place.

Alternatively, as shown in FIGS. 14A-14C, a tissue capture device 450 can include an elongate tubular member 452 terminating with a cutting surface 454 as well as a capture subassembly 456 with a vial 458 attached to a proximal end of the tubular member 452. For ease of manipulation, the elongate tubular member 452 can rotate with respect to the capture subassembly 456 to engage and sever targeted biological substances. Although this embodiment shares features with the immediately preceding embodiment both in structure and use, here a filter is configured within the capture subassembly 456 to effect registering captured material. In this way, captured material can be displaced from the interventional site, and a larger volume of material can be collected.

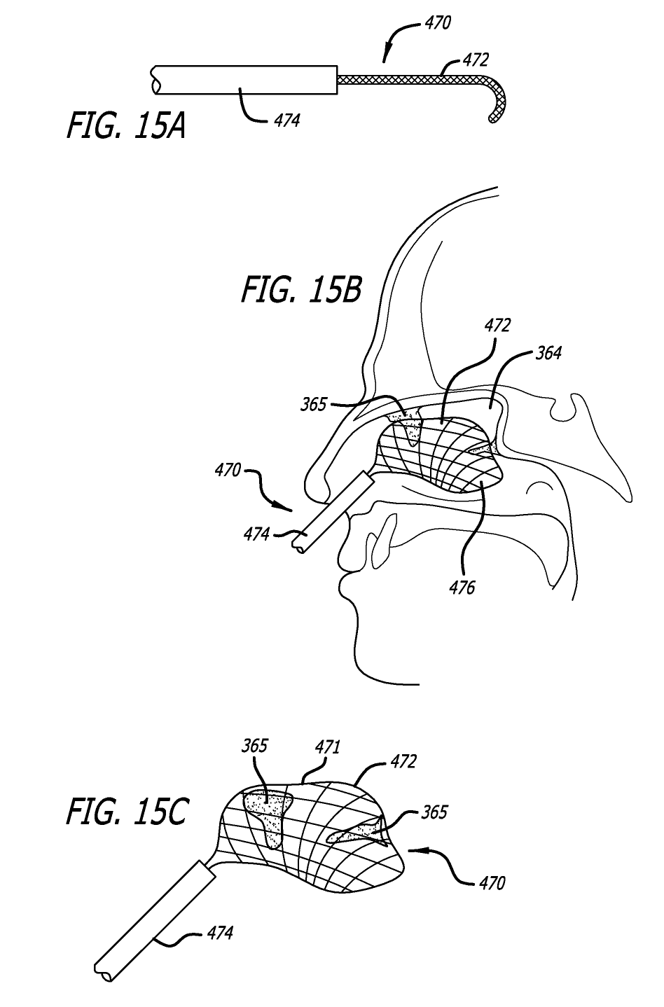

A capture screen device 470 such as that depicted in FIGS. 15A-15C can also be employed to sever and collect biological substances targeted for removal from the paranasal sinuses. As with the previously described embodiments, this device can be used while applying a suctioning force as the capture screen device 470 is used to collect targeted tissues independently of the use of suction. Moreover, as before, this device can incorporate a balloon or other dilation of sinus anatomy and it can form part of a guide catheter and/or be deployable within the same or over a guidewire. The presently contemplated capture screen devices 470 includes an expandable and collapsible screen 472 that is longitudinally translatable within a generally tubular tube 474. Again, the tube 474 can be attached to a suctioning subassembly as desired. Moreover, a balloon (not shown) can be configured within the screen 472 to aid in expanding the screen or contribute to dilation of sinus anatomy.

In use, the capture screen device 470 is placed within a nasal cavity or paranasal sinuses at the interventional site. Once placed as desired, the screen device 472 is advanced beyond the distal end of the tubular member 474 and either permitted to self expand or is expanded by way of opposing motion of members defining the screen 472 (such as a pull wire attached to a distal end of the screen, not shown). Material to be severed is captured between crossing struts 476 of the screen structure 472 when the screen 472 is expanded. Both manipulation of the screen 472 and/or the subsequent withdrawal and collapse of the screen within the tubular member 474 accomplishes cutting the targeted biological material from within the sinus and collection of the material within an interior of the screen. The treatment is completed upon removal of the screen loaded with the collected material from the interventional site. Multiple collection steps can be repeated as necessary to complete the desired treatment.

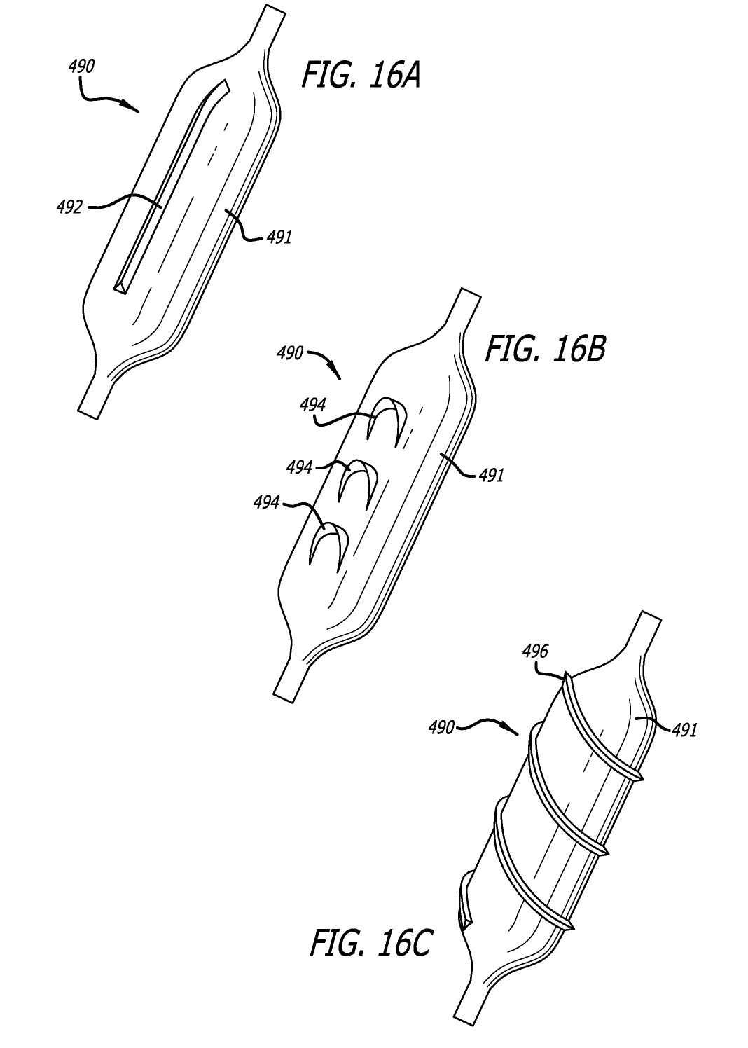

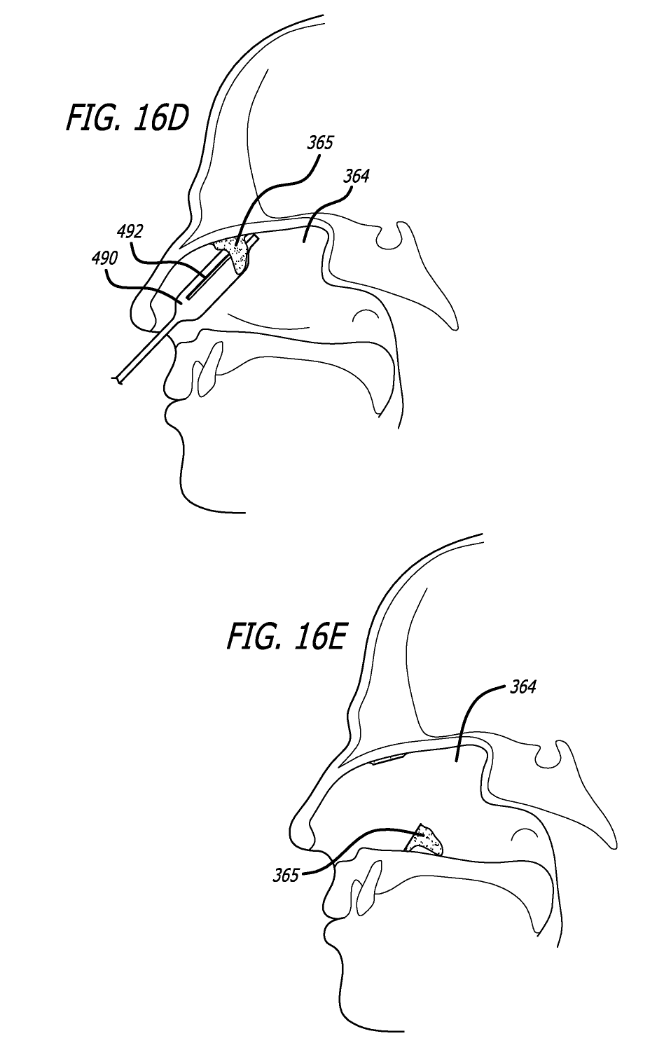

Turning now to FIGS. 16A-16E, another approach to therapies involving biological tissue or substance capture and removal is presented. A balloon catheter 490 including a balloon 491 having an outer surface configured with cutting elements is contemplated for this purpose. Such devices can be advanced over a guidewire and within a guide catheter as disclosed above and can further be used along with suctioning or other capture approaches to remove severed tissue along with or independent from independent balloon dilation of the sinus anatomy. In one approach, the cutting balloon 490 can include one or more blades 492 extending longitudinally along the balloon body 491 (FIG. 16A). The blades can also assume a scooper design 494 or a helical pattern 496 as shown in FIGS. 16B and 16C. To provide treatment at an interventional site, the balloon catheter 470 is expanded to fully expose the blades 492, 494, 496. Manipulating the balloon 490 so that the blades 492, 494, 496 engage and sever the targeted tissue 365 results in dislodging the tissue from walls defining the paranasal cavity. The severed tissue 365 can then be subsequently removed by flushing or by employing a suctioning member (not shown).

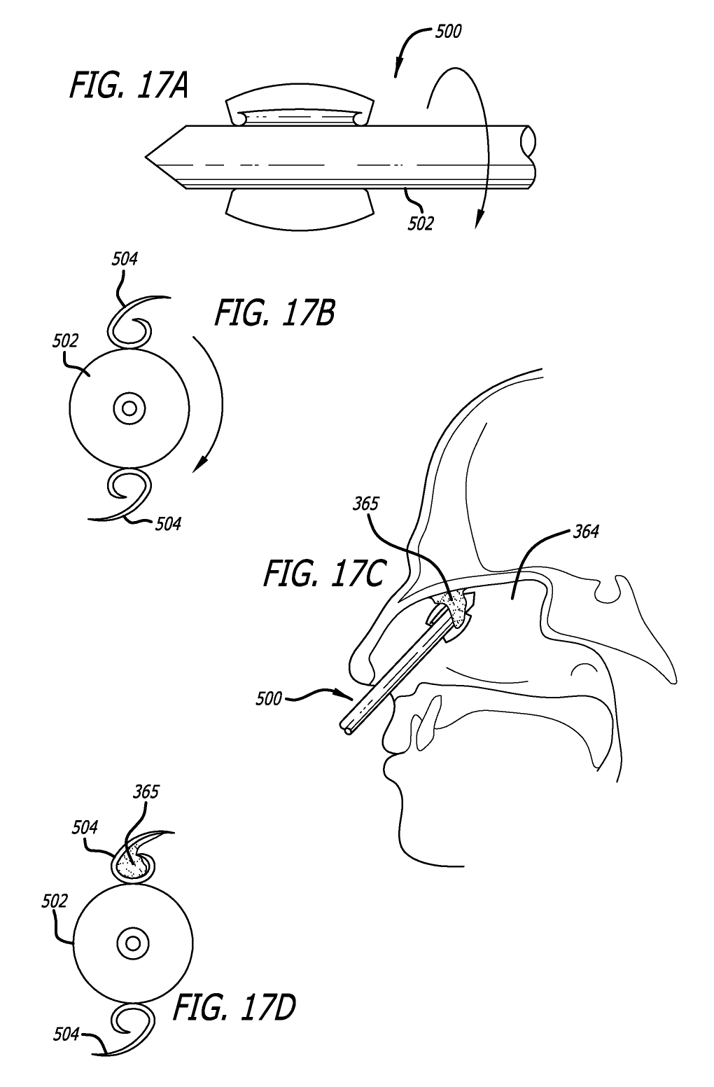

In an alternate approach, the severing of target tissue can be achieved employing a spin cutter device 500 (See FIGS. 17A-17D). Here as well, the spin cutter device 500 can be employed with a dilation catheter for opening paranasal sinus cavities and the device can be advanced within a guide catheter and/or over a guidewire. As shown, the spin cutter device 500 includes an elongate generally cylindrical member 502 having an external surface configured with cutting blades 504 having a curved, rolled profile defining a tissue retention feature. Once placed as desired within sinus anatomy, the spin cutter 500 is rotated so that the cutting blades 504 both cut and capture tissue within its rolled structure. The device can be reused as necessary and then withdrawn from the site employing a sheath so as to avoid trauma to tissue in the area.

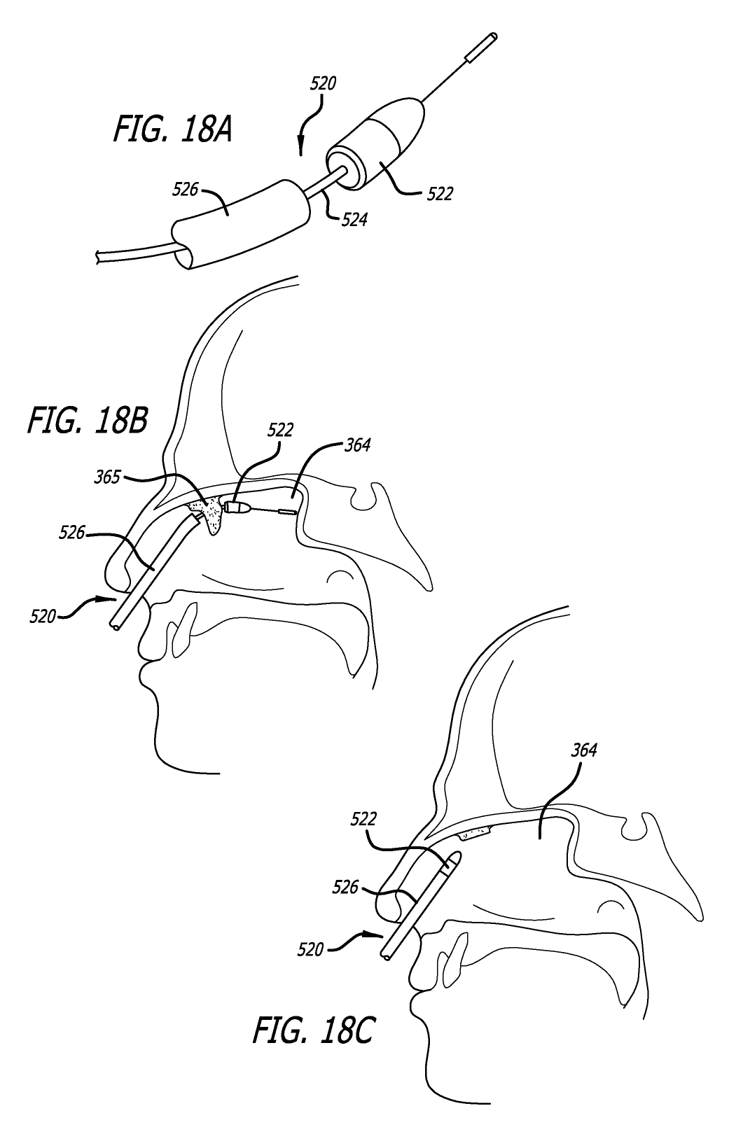

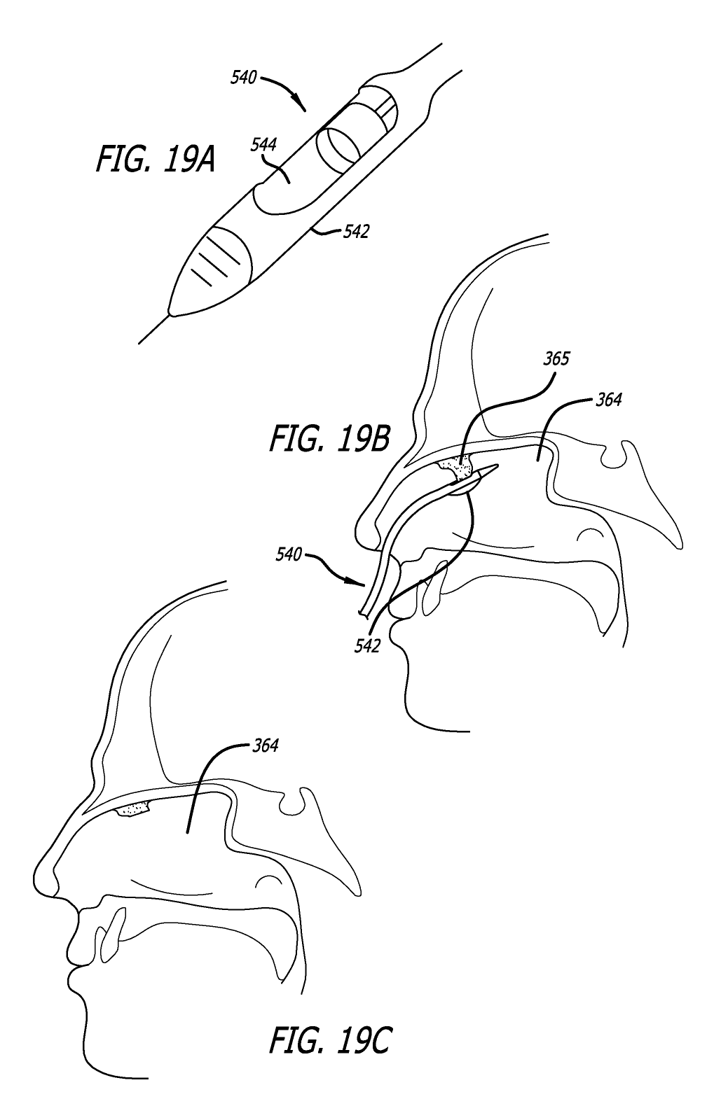

Yet further approaches to tissue collection and removal are depicted in FIGS. 18A-18C and 19A-19C. Such further approaches can be conducted independently from or in combination with one or more of balloon dilation of sinus or nasal cavity anatomy, suctioning pressure, over guidewire advancement and guide catheter introduction. As shown in FIGS. 18A-18C, a back cutter device 520 can be employed to capture biological substances 365 targeted for removal from sinuses. The back cutter device 520 can include a cone 522 supported on a longitudinal member 524, each of which are translatable with request to a generally tubular collection sleeve 526. In one embodiment, the longitudinal member 524 slides within an interior of the sleeve 526 and the cone 522 is sized to be received within a distal end of the sleeve 526. It is contemplated that one or both of the proximal end of the cone 522 and a distal end of the sleeve 526 can include structure for cutting biological substances typically found within sinuses. In this regard, as before, structure defining both the cone 522 and sleeve 526 can include a sharp angle about the center or a portion of a circumference of the cone 522 and sleeve 526. To achieve dissection, targeted matter 365 can be arranged between the cone 522 and the sleeve 526 and the longitudinal member 524 can be rotated to cause the cutting surface of the cone 522 to cut the targeted matter 365. Additionally, the sleeve 526 can also be rotated to cut the targeted matter 365. Once it is believed that a sufficient dissection has occurred, the longitudinal member 524 can be drawn proximally to capture dissected material between the cone 522 and sleeve 526.

In a related embodiment (FIGS. 19A-19C), a balloon and cutter device 540 is configured to cut and capture biological tissue 365 in paranasal sinuses and/or the nasal cavity. Here, the device 540 includes a distal portion that is configured with both a laterally extending expandable balloon as well as an open window 544 positioned on an opposite side of the device from the balloon 542. Configured within the window 544 is a cup-shaped cutter 546, a proximal end of which is connected to a manipulation member 548 extending to an operator. The balloon portion 542 of the device can be employed to open ostia or other spaces within the sinuses or to anchor the device for tissue collection. Tissue collection can occur by placing the window 544 over tissue or other substances to be cut and removed. The cup-shaped cutter 546 can then be rotated and/or advanced against the material to cut and capture the same. The captured material can then be removed from the site by withdrawing the balloon and cutter device from the patient.

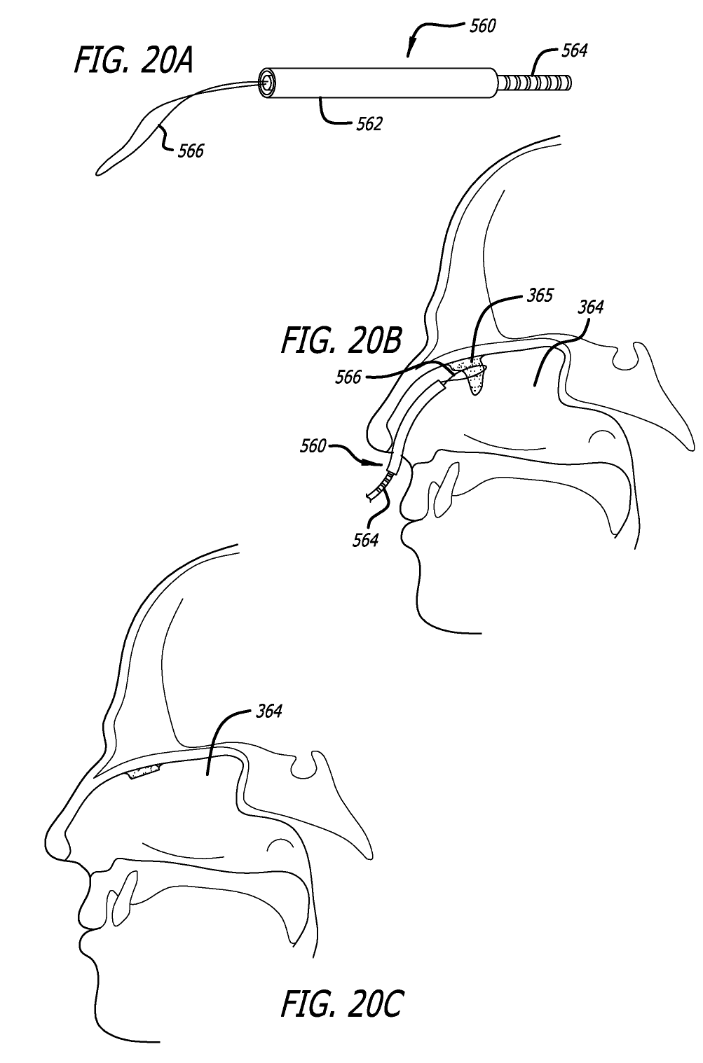

A spinning shaped cutter device 560 such as that depicted in FIGS. 20A-20C also can be used as previously described to access and then be placed adjacent tissue identified for removal from the sinuses. This device is provided with a generally tubular body 562 sized to receive a scored, elongate member 564, the terminal distal end of which includes a shaped cutter 566. In one approach, the cutter 566 is highly flexible and through the manipulation of the elongate member 564, the cutter 566 is rotated to cut tissue. The depth of advancement of the shaped cutter 566 is monitored by noting the position of the markings on the elongate member 564 relative to a proximal end of the tubular body 562. Similar scoring of other embodiments of disclosed therapy devices can be employed to monitor device placement. Additionally, direct or remote viewing technologies such as endoscopy and/or fluoroscopy can be used to aid in tissue dissection and capture.

Yet another approach to sinus therapy (FIGS. 21A-21C) can involve employing a high pressure fluid delivery system 580 to dislodge material from within a patient's nasal cavity 364 and/or paranasal sinus. The system 580 can also be equipped to suction dislodged material from the therapy site through a tubular body 582 as well as a balloon 584 that can be used to stabilize the system or to dilate ostia or other sinus cavities.

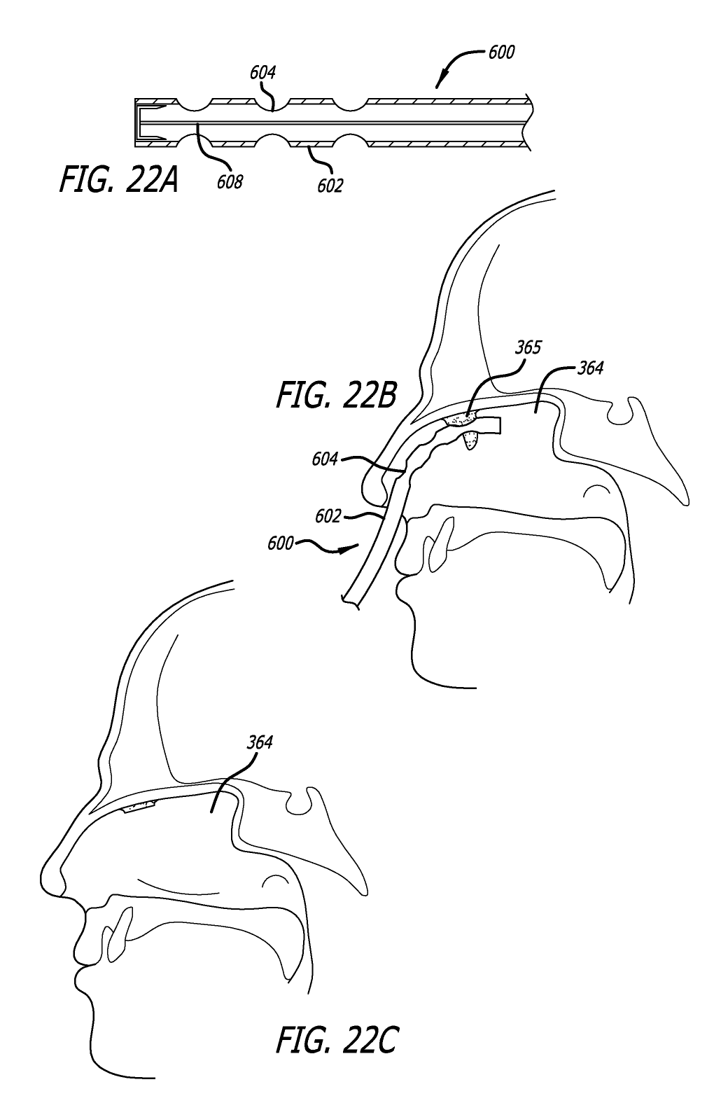

The therapy device 600 can further be defined by a generally tubular member 602 with a plurality of openings 604 formed therein (See FIGS. 22A-22C). In this application, where it has been found to be difficult to cut through hard tissue or bone, a cutter 606 is configured to be responsive to or apply ultrasonic spinning or other oscillary motion to improve cut-ability. That is, motion such as spinning or vibration can induce micromotions that help eliminate friction and further augment cutting ability. The cutter 600 can further be withdrawn as desired to cut tissue adjacent or extending within the holes 604 formed in the device body 602.

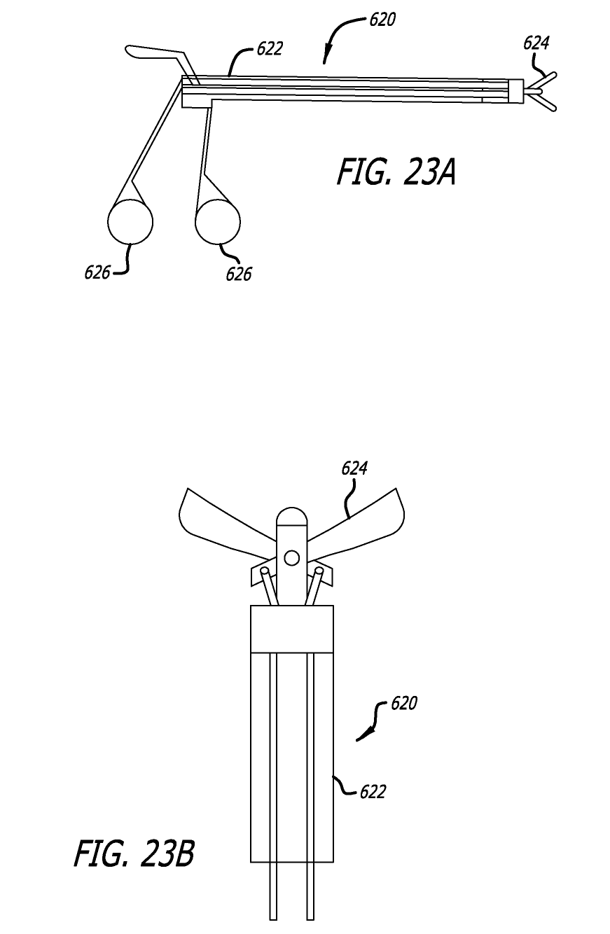

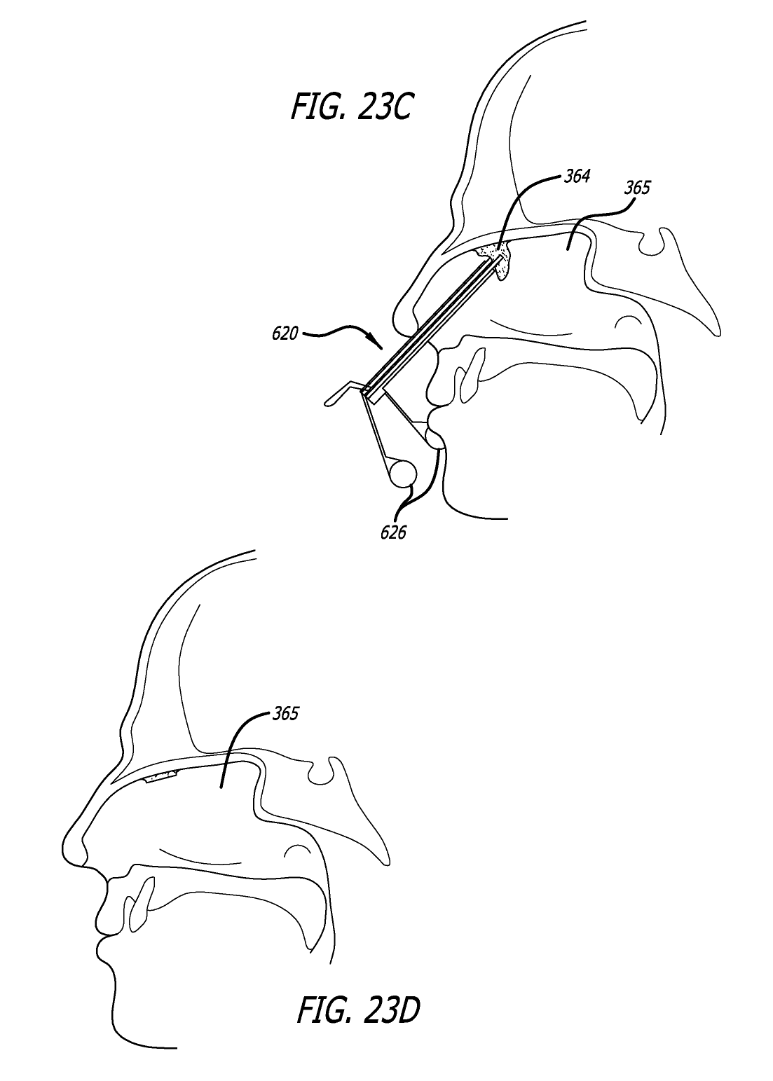

In still yet another approach (FIGS. 23A-23D), in combination with balloon dilation of sinus anatomy, a forceps grasping device 620 is provided to engage and remove tissue from within a paranasal sinus, a paranasal sinus opening and/or a nasal cavity. The device 620 can include a reusable handle 622 and replaceable grasper structure 624. The device can also be entirely disposable or reusable. Moreover, the forceps grasper 620 device may be sized and shaped to be placed through a guide member (not shown). In any event, proximally located finger receptacles 626 are conveniently placed so that tissue extraction can be accomplished remotely.

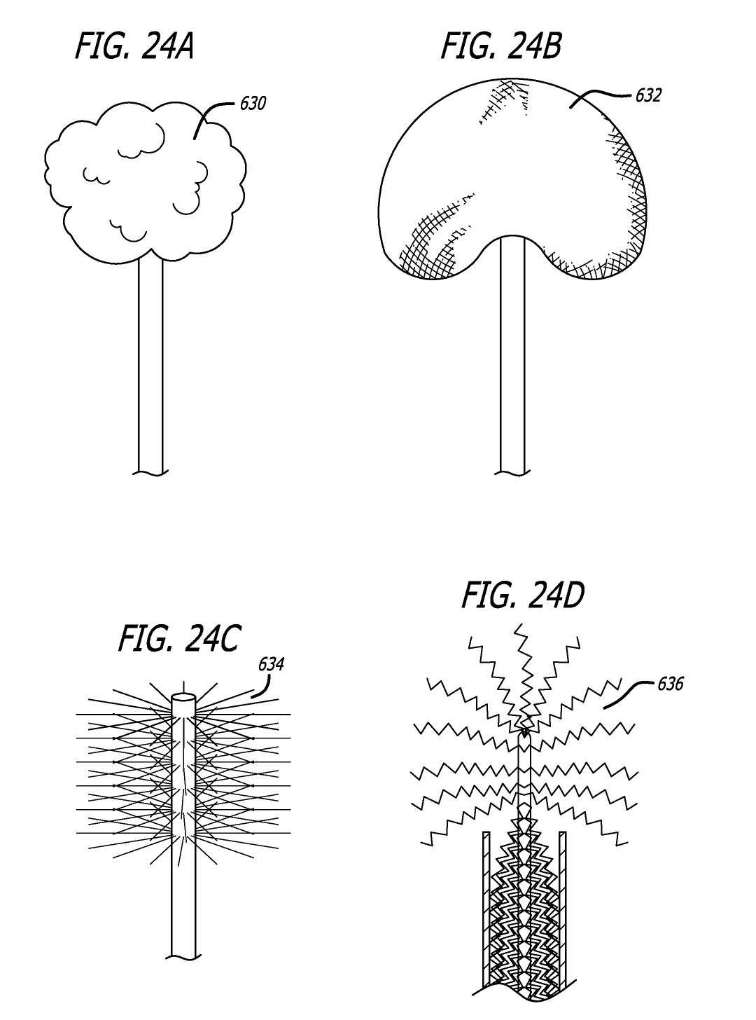

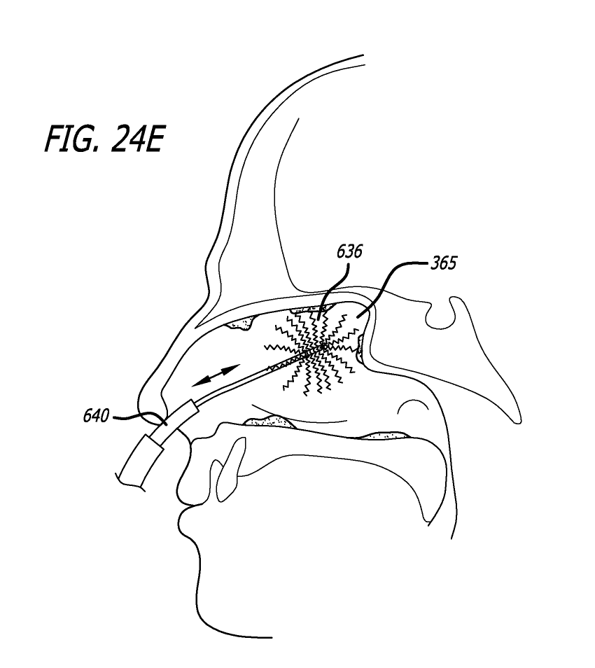

Various approaches to mechanically scrubbing, cleaning and swabbing paranasal cavities are presented in FIGS. 24A-24E. Thus, rather than using sharp blade-like objects, cotton swabs 630 (FIG. 24A), cloth members 632 (FIG. 24B) or a fine brush 634 are contemplated for treating paranasal sinuses. Also contemplated is the use of a wire scrubber 636 depicted in FIG. 24D. For each of these objects, the scrubbing or swabbing members can be advanced through a sheath 640 and permitted to self-expand to a configuration suited for effectively engaging surfaces internal to the paranasal sinuses. Dislodged material can be withdrawn from the therapy site directly by the swabbing/scrubbing device as well as via other disclosed methods (i.e. suctioning).

The removal device may include any of those described above. In various embodiments, the removal device may be advanced through a guide, as shown, or may be advanced on its own, without using a guide. In various embodiments, an endoscope may be used to view all or some of the procedure. In one embodiment, a variable degree of view endoscope may be used, such as a swing prism endoscope described in U.S. patent application Ser. No. 12/502,101, entitled “Swing Prism Endoscope,” the full disclosure of which is hereby incorporated by reference.

In some embodiments, the tissue removal device may include a balloon dilation catheter, for example mounted on a catheter shaft that houses a tissue cutter. Thus, in some embodiments, the dilating and tissue removal functions may be achieved by the same device. In some embodiments, this combination device may be advanced over or through a guide, while in alternative embodiments it may not require a guide.

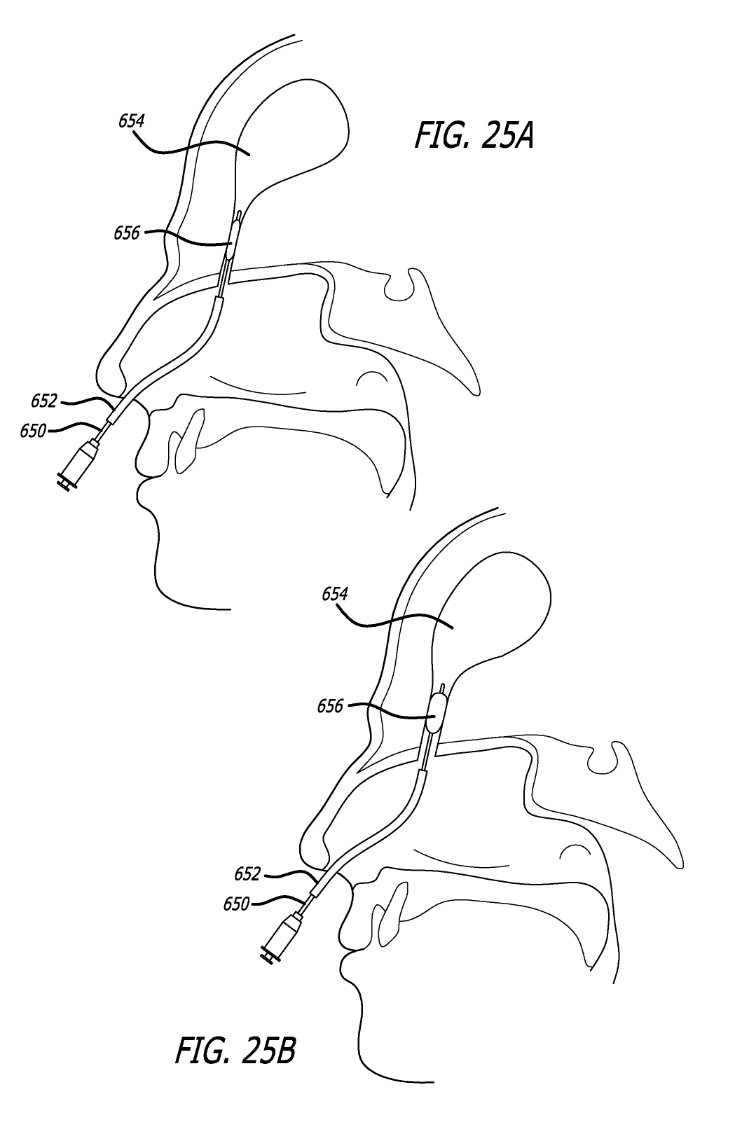

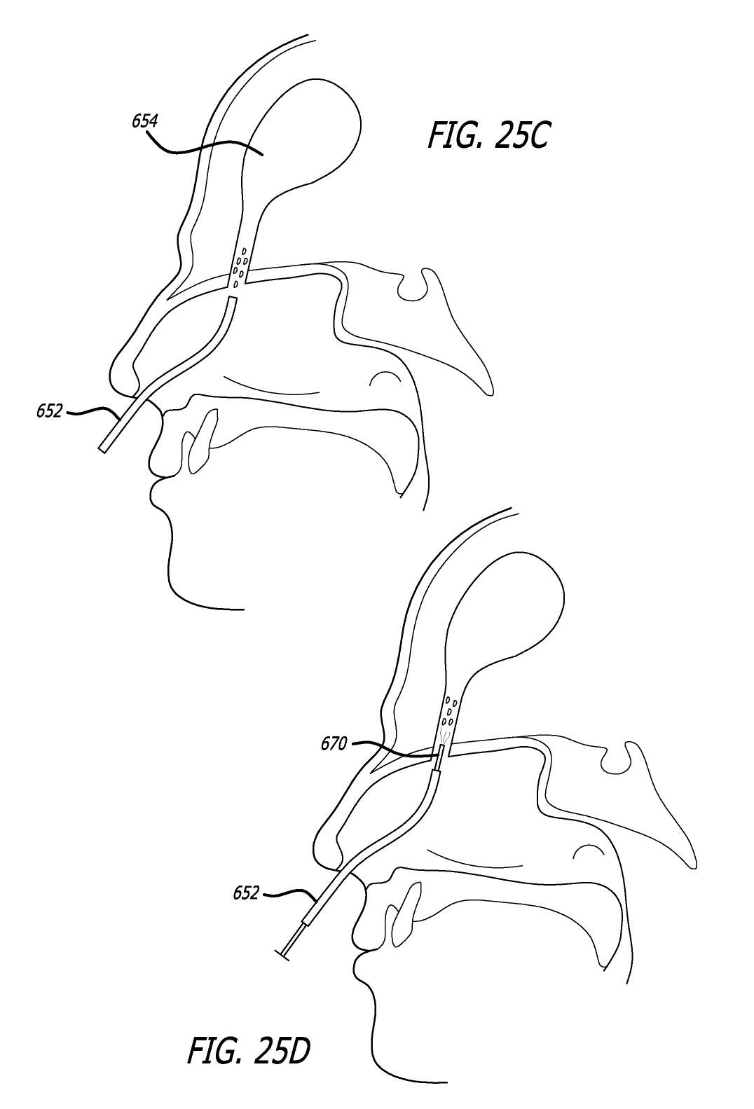

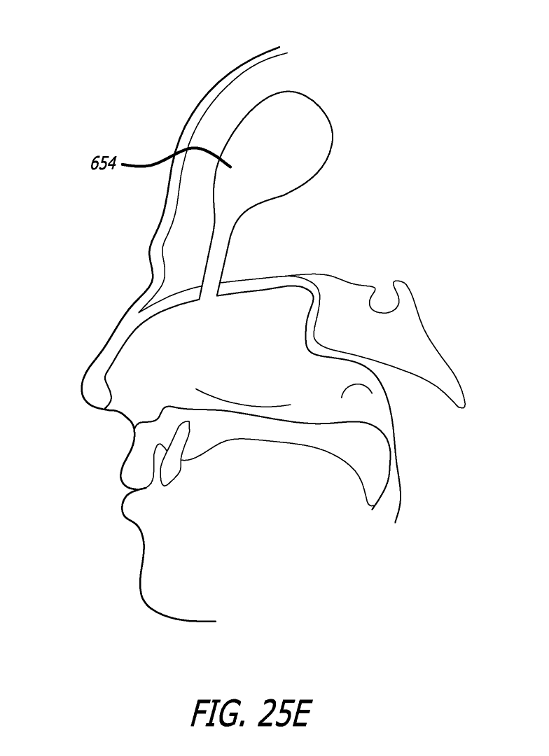

The method shown in FIGS. 25A-25E may also be applied to other paranasal sinuses, such as the maxillary, sphenoid and ethmoid sinuses. Although the ethmoid sinuses typically do not have one discrete, natural opening as the other sinuses do, some combination of dilation and tissue removal may be used in some embodiments to treat the ethmoid sinuses and/or areas of the nasal cavity such as the osteomeatal complex. Here, a balloon catheter 650 is advanced through a guide catheter 652 to within an ostium or outflow tract 653 leading to a frontal sinus 654. The balloon 656 of the balloon catheter is expanded (FIG. 25B) to open the ostium or outflow tract. The balloon catheter 650 is then removed leaving the guide catheter in place (FIG. 25C). Bone fragments or other biological substances 660 may remain at the site. Thus, a tissue removal device 670 such as those described above can be advanced to the site through the guide 652 (FIG. 25D). Once there, the fragments can be removed to thereby leave an expanded, clear outflow tract and ostium.

In various alternative embodiments, the devices, systems and methods described above may be used for diagnosing or treating other conditions caused by narrowing or blockage of structures in the ear, nose, throat or mouth. Also in various embodiments, devices described herein such as catheters may comprise one or more lumens such as end-to-end lumens, zipper lumens, rapid exchange lumens, parallel lumens surrounded by a jacket, and the like.

The above description provides a number of examples and embodiments, but various additions, deletions, alterations and modifications may be made to these examples and embodiments without departing from the intended spirit and scope of the present invention. For example, any element or attribute of one embodiment or example may be incorporated into or used with another embodiment or example, unless to do so would render the embodiment or example unsuitable for its intended use. All reasonable additions, deletions, modifications and alterations are to be considered equivalents of the described examples and embodiments and are to be included within the scope of the following claims.

We claim:

1. A method for treating a paranasal sinus in a patient, the method comprising:
advancing a dilating device along a guide device into the patient's head;
dilating an opening into the paranasal sinus, using the dilating device;
removing the dilating device from the head while leaving the guide in the patient's head;
advancing a tissue removal device along the guide into the paranasal sinus; and
removing tissue from at least one of the paranasal sinus, a different paranasal sinus, or a nasal cavity of the patient using the tissue removal device.

2. The method of claim 1, wherein the paranasal sinus is selected from the group consisting of maxillary, sphenoid, frontal and ethmoid sinuses.

3. The method of claim 1, wherein the guide device is selected from the group consisting of a guide catheter with a lumen, a guidewire and a combination of a guide catheter and a guidewire.

4. The method of claim 1, wherein the dilating device comprises a balloon catheter.

5. The method of claim 1, wherein dilating the opening involves dilating a paranasal ostium.

6. The method of claim 1, wherein dilating the opening involves dilating a manmade opening.

7. The method of claim 1, wherein removing the tissue comprises:
suctioning the tissue into the tissue removal device; and
cutting off the suctioned tissue using a cutter of the tissue removal device.

8. The method of claim 1, wherein removing the tissue comprises removing tissue from the opening into the paranasal sinus.

9. The method of claim 1, wherein removing the tissue comprises removing tissue from within the paranasal sinus.

10. The method of claim 1, wherein dilating the opening comprises dilating at least one of a frontal sinus ostium and a frontal sinus outflow tract, and wherein removing the tissue comprises removing tissue from the frontal sinus outflow tract.

11. The method of claim 1, wherein dilating the opening comprises dilating a natural paranasal sinus ostium of the paranasal sinus, and wherein removing the tissue comprises removing bone fragments.

12. The method of claim 1, wherein dilating the opening comprises dilating a natural paranasal sinus ostium of the paranasal sinus, and wherein removing the tissue comprises removing at least part of an ethmoid sinus.

13. The method of claim 1, wherein removing tissue involves removing a type of tissue selected from the group consisting of polyps, mucosal tissue, cysts, bone fragments, bone and mucocysts.

14. The method of claim 1, wherein removing tissue involves employing apparatus selected from the group consisting of a morcellator, a snare, a combined snare/cutter, a filter, a radiofrequency cutting and/or coagulating device, a contractable mesh device, a balloon configured with blades, a bone cutter assembly, a tube with openings in combination with a cutter, a high pressure spray device, and a forceps-grasper assembly.

15. A method for treating a paranasal sinus in a patient, the method comprising:
advancing a guide catheter into the patient's head;
advancing a guidewire through the guide catheter and through an opening into the paranasal sinus;
advancing a balloon dilation catheter over the guidewire through the guide to position a balloon of the catheter in the opening;
dilating the opening by expanding the balloon;
removing the balloon, leaving at least the guidewire in place;
advancing a tissue removal device over the guidewire into the patient's head;
removing tissue from at least one of the paranasal sinus, a different paranasal sinus and a nasal cavity of the patient with a tissue removal device; and
removing the tissue removal device and the guidewire from the patient's head.

16. A method for treating a paranasal sinus in a patient, the method comprising:
advancing a guide device into the patient's head;
advancing a tissue dilation and removal device along the guide device to position a dilator of the device in an opening into the paranasal sinus;
dilating the opening by expanding the dilator;
removing tissue from the paranasal sinus using the tissue dilation and removal device; and
removing the tissue dilation and removal device and the guide device from the patient's head.

17. A system for treating a paranasal sinus in a patient, the system comprising:
a guide device for guiding one or more devices into the patient's head to treat the paranasal sinus;
a dilation device for dilating an opening into the paranasal sinus and configured for passage along the guide device; and
a tissue removal device for removing tissue from inside the paranasal sinus and configured for passage along the guide device.

18. The system of claim 17, wherein the paranasal sinus is selected from the group consisting of maxillary, sphenoid, frontal and ethmoid sinuses.

19. The system of claim 18, wherein the guide device comprises a guide catheter with a lumen.

20. The system of claim 19, wherein the guide device further comprises a guidewire.

21. The system of claim 19, wherein the dilating device is a balloon catheter.

22. The system of claim 20, wherein the opening is a paranasal ostium.

23. The system of claim 20, wherein the opening is a manmade opening.

24. The system of claim 20, wherein the tissue removal device includes suction substructure.

25. The system of claim 20, wherein the tissue removal device includes a cutter.

26. The system of claim 20, wherein the tissue removal device is configured to remove tissue selected from the group consisting of polyps, mucosal tissue, cysts, bone fragments, bone and mucocysts.

27. The system of claim 20, wherein the tissue removal device includes a snare.

28. The system of claim 20, wherein the tissue removal device is configured to deliver RF energy.

29. A system for treating a paranasal sinus in a patient, the device comprising:
a guide device for guiding one or more devices into the patient's head to treat the paranasal sinus; and
a combined tissue dilation and removal device for dilating an opening into the paranasal sinus and removing tissue from the paranasal sinus, wherein the tissue dilation and removal device is configured for passage along the guide device.
